SEQUENCE LISTING (39 sequences)

SEQ ID NO: 1 - DNA, 16 bp, synthetic construct
  source 1..16: mol_type = other DNA
atttcattcgacaccc

SEQ ID NO: 2 - DNA, 16 bp, synthetic construct
  source 1..16: mol_type = other DNA
ggtgcagcctcattgt

SEQ ID NO: 3 - DNA, 15 bp, synthetic construct
  source 1..15: mol_type = other DNA
gtttgagtgaaggcg

SEQ ID NO: 4 - DNA, 18 bp, synthetic construct
  source 1..18: mol_type = other DNA
gtccaacccttagtgcgt

SEQ ID NO: 5 - DNA, 18 bp, synthetic construct
  source 1..18: mol_type = other DNA
ttcggccagagtttaatg

SEQ ID NO: 6 - DNA, 14 bp, synthetic construct
  source 1..14: mol_type = other DNA
cgtgcagcttgcgt

SEQ ID NO: 7 - DNA, 15 bp, synthetic construct
  source 1..15: mol_type = other DNA
gtaccctgcgatcag

SEQ ID NO: 8 - DNA, 16 bp, synthetic construct
  source 1..16: mol_type = other DNA
actccagctgcgtagt

SEQ ID NO: 9 - DNA, 16 bp, synthetic construct
  source 1..16: mol_type = other DNA
ctgcttgcatagggag

SEQ ID NO: 10 - DNA, 17 bp, synthetic construct
  source 1..17: mol_type = other DNA
ggaatcttggtgcgact

SEQ ID NO: 11 - DNA, 18 bp, synthetic construct
  source 1..18: mol_type = other DNA
atttcattcgacacccgg

SEQ ID NO: 12 - DNA, 15 bp, synthetic construct
  source 1..15: mol_type = other DNA
tcgcagcctcattgt

SEQ ID NO: 13 - DNA, 16 bp, synthetic construct
  source 1..16: mol_type = other DNA
gtttgagtgaagggtc

SEQ ID NO: 14 - DNA, 15 bp, synthetic construct
  source 1..15: mol_type = other DNA
caacccttagtgcgt

SEQ ID NO: 15 - DNA, 19 bp, synthetic construct
  source 1..19: mol_type = other DNA
ttcggccagagtttaatgc

SEQ ID NO: 16 - DNA, 14 bp, synthetic construct
  source 1..14: mol_type = other DNA
gttgcagcttgcgt

SEQ ID NO: 17 - DNA, 17 bp, synthetic construct
  source 1..17: mol_type = other DNA
gtaccctgcgatcagac

SEQ ID NO: 18 - DNA, 14 bp, synthetic construct
  source 1..14: mol_type = other DNA
tccagctgcgtagt

SEQ ID NO: 19 - DNA, 17 bp, synthetic construct
  source 1..17: mol_type = other DNA
ctgcttgcatagggagg

SEQ ID NO: 20 - DNA, 16 bp, synthetic construct
  source 1..16: mol_type = other DNA
gaatcttggtgcgact

SEQ ID NO: 21 - DNA, 33 bp, synthetic construct
  source 1..33: mol_type = other DNA
atttcattcgacacccgggtcgagcctcattgt

SEQ ID NO: 22 - DNA, 18 bp, synthetic construct
  source 1..18: mol_type = other DNA
atttcattcgacacccgg

SEQ ID NO: 23 - DNA, 15 bp, synthetic construct
  source 1..15: mol_type = other DNA
tcgcagcctcattgt

SEQ ID NO: 24 - DNA, 19 bp, synthetic construct
  source 1..19: mol_type = other DNA
atttcattcgacacccggc

SEQ ID NO: 25 - DNA, 11 bp, synthetic construct
  source 1..11: mol_type = other DNA
agcctcattgt

SEQ ID NO: 26 - DNA, 16 bp, synthetic construct
  source 1..16: mol_type = other DNA
atttcattcgacaccc

SEQ ID NO: 27 - DNA, 21 bp, synthetic construct
  source 1..21: mol_type = other DNA
atttcattcgacacccggcat

SEQ ID NO: 28 - DNA, 18 bp, synthetic construct
  source 1..18: mol_type = other DNA
atttcattcgacacccgg

SEQ ID NO: 29 - DNA, 15 bp, synthetic construct
  source 1..15: mol_type = other DNA
tcgcagcctcattgt

SEQ ID NO: 30 - DNA, 18 bp, synthetic construct
  source 1..18: mol_type = other DNA
gtttgagtgaaggcggtc

SEQ ID NO: 31 - DNA, 15 bp, synthetic construct
  source 1..15: mol_type = other DNA
caacccttagtgcgt

SEQ ID NO: 32 - DNA, 19 bp, synthetic construct
  source 1..19: mol_type = other DNA
ttcggccagagtttaatgc

SEQ ID NO: 33 - DNA, 14 bp, synthetic construct
  source 1..14: mol_type = other DNA
gttgcagcttgcgt

SEQ ID NO: 34 - DNA, 18 bp, synthetic construct
  source 1..18: mol_type = other DNA
gtaccgtgcgaatcagac

SEQ ID NO: 35 - DNA, 15 bp, synthetic construct
  source 1..15: mol_type = other DNA
tccagcttgcgtagt

SEQ ID NO: 36 - DNA, 17 bp, synthetic construct
  source 1..17: mol_type = other DNA
ctgcttgcatagggagg

SEQ ID NO: 37 - DNA, 16 bp, synthetic construct
  source 1..16: mol_type = other DNA
gaatcttggtgcgact

SEQ ID NO: 38 - DNA, 18 bp, synthetic construct
  source 1..18: mol_type = other DNA
cgagttggacaatgatcg

SEQ ID NO: 39 - DNA, 15 bp, synthetic construct
  source 1..15: mol_type = other DNA
cctcccaaggttcct

(19) United States
(12) Patent Application Publication
(10) Pub. No.: US 2026/0275407 A1
(43) Pub. Date: Sep. 17, 2026

(54) DETECTION OF ANALYTES IN CELLS AND TISSUES

(71) Applicant: Singular Genomics Systems, Inc., San Diego, CA (US)

(72) Inventors: Ryan COSTELLO, San Diego, CA (US); Jessey VELASQUEZ, San Diego, CA (US); Andrew PAWLOWSKI, San Diego, CA (US)

(21) Appl. No.: 19/564,081

(22) Filed: Mar. 11, 2026

Related U.S. Application Data
(60) Provisional application No. 63/771,224, filed on Mar. 13, 2025.

(51) Int. Cl. C12Q 1/6841; C12Q 1/6804; C12Q 1/6806; C12Q 1/6823; C12Q 1/6825; C12Q 1/6844

(52) U.S. Cl. CPC C12Q 1/6841; C12Q 1/6804; C12Q 1/6806; C12Q 1/6823; C12Q 1/6825; C12Q 1/6844

(57) ABSTRACT

Disclosed herein, inter alia, are compositions and methods for improved multiplexed detection of biomolecules in cells and tissues.

### CROSS-REFERENCES TO RELATED APPLICATIONS

This application claims the benefit of U.S. Provisional Application No. 63/771,224, filed Mar. 13, 2025, which is incorporated herein by reference in its entirety and for all purposes.

### SEQUENCE LISTING

The Sequence Listing written in file 642001US.xml, created Mar. 3, 2026, which is 35,160 bytes, machine format IBM-PC, MS Windows operating system, is hereby incorporated by reference.

### BACKGROUND

The field of cellular biology has increasingly focused on the identification and quantification of proteins within their native cellular environments. In situ protein detection is essential for understanding the complex interactions of biomolecules within cells and provides critical insights into cellular functions, signaling pathways, and disease mechanisms. The development of molecular techniques has transformed in situ protein detection by integrating nucleic acid-based strategies with traditional protein detection approaches. For example, sequencing-based methods utilizing antibody-oligonucleotide conjugates have introduced new possibilities for high-throughput multiplexed protein analysis. These advancements enable the simultaneous detection of multiple proteins within a single sample, a capability that is particularly valuable in fields such as cancer research, where the expression patterns of numerous proteins can provide diagnostic and prognostic information.

To expand multiplexing capacity, sequencing-based protein detection methods may employ multiple rounds of sequencing primers, enabling batchwise readout of different amplification products. For example, a first circularizable probe may contain a first primer binding site, complementary to a first sequencing primer. Similarly, second and third circularizable probes may contain distinct second and third primer binding sites, complementary to different sequencing primers. Following probe circularization and amplification, sequencing proceeds in multiple rounds, each using a different primer. In the first sequencing round, primer 1 hybridizes and sequences the probe bound to a first nucleic acid molecule. In the second round, primer 2 hybridizes and sequences a probe bound to a second nucleic acid molecule. Likewise, in the third round, primer 3 hybridizes and sequences a third nucleic acid molecule, allowing for the detection of a greater number of targets within a single sample. Despite its advantages in multiplexing, transitioning between sequencing primer rounds introduces challenges, particularly in sequencing read termination and primer removal. Inefficient termination can result in the incorporation of unintended nucleotides, leading to background signal and reduced sequencing accuracy. Disclosed herein, inter alia, are solutions to these and other problems in the art.

### BRIEF SUMMARY

In an aspect is provided a method of detecting polynucleotides, the method including: (i) hybridizing a first sequencing primer to a first polynucleotide, incorporating with a polymerase a first labeled nucleotide into the first sequencing primer, and detecting the first labeled nucleotide; (ii) cleaving the first sequencing primer into a first fragment and a second fragment and removing the fragments; and (iii) hybridizing a second sequencing primer to a second polynucleotide, incorporating with a polymerase a second labeled nucleotide into the second sequencing primer, and detecting the second labeled nucleotide, wherein the first polynucleotide and the second polynucleotide are in a cell or tissue.

According to an aspect of the present disclosure, a method of detecting biomolecules in a cell or tissue is provided. The method includes hybridizing a first oligonucleotide to a first target polynucleotide in the cell or tissue, wherein the first oligonucleotide includes a cleavable site, and detecting a first detectable label associated with the first oligonucleotide. The method further includes cleaving the first oligonucleotide at the cleavable site to produce a first fragment and a second fragment, wherein each of the first fragment and the second fragment has a melting temperature lower than a melting temperature of the first oligonucleotide, and removing the first fragment and the second fragment. The method further includes hybridizing a second oligonucleotide to a second target polynucleotide in the cell or tissue, and detecting a second detectable label associated with the second oligonucleotide.

According to other aspects of the present disclosure, the method may include one or more of the following features. The hybridizing, cleaving, and subsequent hybridizing may occur at substantially the same temperature. The hybridizing, cleaving, and subsequent hybridizing may occur at about 55° C. to about 65° C. The hybridizing, cleaving, and subsequent hybridizing may occur at about 60° C. The first detectable label may be a fluorescent label covalently attached to the first oligonucleotide. The first oligonucleotide may be a sequencing primer and detecting the first detectable label may include binding a labeled nucleotide to the first oligonucleotide with a polymerase and detecting the labeled nucleotide. The first oligonucleotide may be a sequencing primer and detecting the first detectable label may include incorporating a labeled nucleotide into the first oligonucleotide with a polymerase and detecting the labeled nucleotide. The labeled nucleotide may include a reversible terminator moiety and a fluorescent label covalently linked to the nucleotide via a cleavable linker. The method may further include incorporating a plurality of labeled nucleotides into the first oligonucleotide in a plurality of sequencing cycles prior to cleaving. The cleavable site may include a disulfide scissile internucleosidic linkage. The first oligonucleotide may include a C6-S—S linker. The cleavable site may include a deoxyuracil triphosphate (dUTP), a deoxy-8-oxo-guanine triphosphate (d-8-oxoG), a methylated nucleotide, or a ribonucleotide. The first oligonucleotide may include two or more cleavable sites. Cleaving the first oligonucleotide may include contacting the first oligonucleotide with a reducing agent. Cleaving the first oligonucleotide may include contacting the first oligonucleotide with sodium periodate or a nuclease. Cleaving the first oligonucleotide may include contacting the first oligonucleotide with uracil DNA glycosylase (UDG), Formamidopyrimidine DNA Glycosylase (Fpg), or RNase. The method may further include repeating the hybridizing, cleaving, and subsequent hybridizing in series to detect additional biomolecules. The method may further include repeating the hybridizing, cleaving, and subsequent hybridizing at least 3 times in series. The first target polynucleotide and the second target polynucleotide may each be formed via rolling circle amplification of a circularized probe. The circularized probe may be hybridized to an oligonucleotide conjugated to a specific binding agent, wherein the specific binding agent is an antibody, single-chain Fv fragment (scFv), antibody fragment-antigen binding (Fab), affimer, or an aptamer. The circularized probe may include a barcode sequence.

According to another aspect of the present disclosure, a system for detecting biomolecules in a cell or tissue is provided. The system includes a substrate including a channel, wherein the cell or tissue is immobilized within the channel. The system includes a first oligonucleotide including a cleavable site, wherein cleaving the first oligonucleotide at the cleavable site produces a first fragment and a second fragment, each having a melting temperature lower than a melting temperature of the intact first oligonucleotide. The system includes a second oligonucleotide. The system includes a cleaving agent configured to cleave the cleavable site of the first oligonucleotide. The system includes a polymerase. The system includes a plurality of labeled nucleotides. The system is configured to sequentially: (i) hybridize the first oligonucleotide to a first target polynucleotide in the cell or tissue and detect a signal associated with the first oligonucleotide, (ii) cleave the first oligonucleotide at the cleavable site and remove the first fragment and the second fragment, and (iii) hybridize the second oligonucleotide to a second target polynucleotide in the cell or tissue and detect a signal associated with the second oligonucleotide.

According to other aspects of the present disclosure, the system may include one or more of the following features. The system may further include a fluidic system in fluid communication with the channel, wherein the fluidic system is configured to deliver the first oligonucleotide, the second oligonucleotide, the cleaving agent, the polymerase, and the plurality of labeled nucleotides to the channel. The system may further include an imaging system configured to detect the signal associated with the first oligonucleotide and the signal associated with the second oligonucleotide. The imaging system may include a fluorescence excitation source. The cleaving agent may include a reducing agent. The reducing agent may include tris(2-carboxyethyl)phosphine (TCEP) or tris(3-hydroxypropyl)phosphine (THPP). The substrate may include a glass slide. The substrate may include a flow cell. The system may be configured to maintain a substantially constant temperature during the hybridizing, cleaving, and subsequent hybridizing steps.

According to another aspect of the present disclosure, a method of detecting a plurality of proteins in a cell or tissue is provided. The method includes contacting the cell or tissue with a plurality of circularizable probes and forming a plurality of oligonucleotide complexes, wherein the cell or tissue includes the plurality of proteins and each protein includes an oligonucleotide, and forming an oligonucleotide complex includes binding a circularizable probe to the oligonucleotide. The method includes circularizing each circularizable probe to form a plurality of circular polynucleotides, wherein each circular polynucleotide includes a primer binding sequence and a barcode sequence. The method includes amplifying the circular polynucleotides by rolling circle amplification to generate a plurality of amplification products. The method includes hybridizing a first sequencing primer to a first subset of the amplification products, wherein the first sequencing primer includes a cleavable site, incorporating with a polymerase a first labeled nucleotide into the first sequencing primer, and detecting the first labeled nucleotide to identify a first subset of target proteins. The method includes cleaving the first sequencing primer at the cleavable site to produce a first fragment and a second fragment, and removing the first fragment and the second fragment. The method includes hybridizing a second sequencing primer to a second subset of the amplification products, incorporating with a polymerase a second labeled nucleotide into the second sequencing primer, and detecting the second labeled nucleotide to identify a second subset of target proteins.

### BRIEF DESCRIPTION OF THE DRAWINGS

FIGS. 1A-1B demonstrates ineffective blocking between rounds of sequencing. A representative series of images of kidney is shown in FIG. 1A. Briefly, following sample preparation and amplification, a first image of the kidney is obtained (i), followed by incorporation of a labeled nucleotide (ii). The labeled nucleotide is incorporated into a sequencing primer bound to the amplification product. Following cleavage and incorporation of a terminated nucleotide (e.g., ddNTP), a third image is obtained (iii). One would expect that after incorporating an irreversibly terminated nucleotide incapable of extending, that upon exposure to additional labeled nucleotides nothing would be incorporated. However, as shown in FIG. 1A panel (iv), labeled nucleotides are detected, indicating the nucleotides are still binding to the polymerase complex during imaging. When sequencing over multiple rounds, for example 5 different rounds of sequencing in tonsil, biomolecules are illuminated and detected in every round (FIG. 1B). A method that enables efficient primer removal while maintaining sample integrity is critical for improving the accuracy, scalability, and reproducibility of this sequencing-based protein detection platform.

FIGS. 2A-2B illustrates a primer containing a cleavable site, SS—C6. Two portions of the primer are connected via a disulfide linkage, as illustrated in FIG. 2A. Following cleavage, the oligonucleotide primer having a first melting temperature is split in two distinct portions, FIG. 2B, each of which have a lower melting temperature. The entire primer is not shown, but rather two nucleotides linked via a cleavable site, referred to as SS—C6. See for example, Table 3, which shows the 5′ and 3′ arms, respectively, which are linked via the cleavable site.

FIG. 3. The performance of the cleavable primers was assessed by measuring residual signal in post-block extension to evaluate the efficiency of primer cleavage and removal. Primers S1c+15, S1C+11, as well as the double-cleavage primer (S1de), exhibited little to no observable signal in post-block extension, indicating that cleavage and primer removal were highly efficient, relative to the control S1nc.

FIG. 4 shows multiplex detection of CD3, CD20, ATPase, HLADR, CD8, and CD45RA in tonsil tissue, as detected via two temporally separated rounds of detection.

FIGS. 5A-5C illustrate a round of isothermal detection. For example, two independent sequencing primers are bound to distinct target polynucleotides. Each sequencing primer includes one or more cleavable sites, depicted as an “X” in FIG. 5A. A labeled nucleotide is incorporated into each respective sequencing primer, FIG. 5B, and the cleavable site and the label are cleaved as shown in FIG. 5C. The two fragments have a reduced melting temperature and de-hybridize from the target polynucleotide. The process illustrated in FIGS. 5A-5C may be performed at substantially the same temperature (e.g., the temperature is maintained at about 60° C.). Additional rounds of sequencing may subsequently be performed in a similar manner.

FIG. 6 provides an illustration of a probe conjugate described herein (e.g., a specific binding agent described herein). The embodiment depicted in FIG. 6 includes an antibody covalently attached to two oligonucleotides via independent bioconjugate linkers. The oligonucleotide provides a sequence that is associated with the antibody, and so when the sequence of the oligonucleotide is inferred or detected the identity of the antibody and thus the target protein of interest is identified.

FIG. 7 provides an illustration of the sequential collection of information to inform on the structure of a cell and/or tissue. Spectrally distinct dyes are used in the first set, and optionally reused in subsequent sets. For example, the first set includes Alexa Fluor® 532 (emission: 532 nm), Alexa Fluor® 594 (emission: 594 nm), Alexa Fluor® 647 (emission: 647 nm), and Alexa Fluor® 680 (emission: 680 nm) to illuminate cellular components, such as proteins, the Golgi Apparatus, endoplasmic reticulum, actin, lysosomes, and/or specific cell surface receptors of a cell. Following cleavage and removal of the fluorophores, the second set of targeting molecules are incubated with the sample cell. The second set can then illuminate the nucleus, nucleoli, mitochondria, nuclear envelop, cell surface receptors, and plasma membrane. The sequential addition of labels can continue for N cycles providing additional information about the cell. The resulting images may be computationally processed and overlaid to provide a composite image of the cell and/or tissue.

### DETAILED DESCRIPTION

The aspects and embodiments described herein relate to detection of multiple targets in a biological sample. The methods and systems described herein provide significant advantages over conventional multiplexed detection approaches. Unlike traditional immunofluorescence and immunohistochemistry techniques, which are constrained by spectral overlap and typically limited to detecting only four or five targets simultaneously, the cleavable primer-based sequencing approach enables detection of substantially more targets within a single sample. The controlled fragmentation of sequencing primers following each detection round eliminates interference from residual primers, thereby reducing background signal and improving sequencing accuracy compared to methods that rely on photobleaching or harsh denaturing conditions. Furthermore, by maintaining substantially isothermal conditions throughout the hybridization, cleavage, and detection steps, the methods preserve sample integrity and simplify workflow implementation. The integration of rolling circle amplification with barcode-based target identification provides robust signal amplification while maintaining spatial resolution, addressing limitations of cyclic imaging approaches that can damage samples through repeated staining and bleaching cycles. These features collectively support scalable, high-throughput multiplexed analysis suitable for complex biological samples in spatial biology applications.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. In embodiments, the cleavable site or scissile site may be include a disulfide linker and is cleaved with a cleaving agent.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing.

The term “orthogonal detectable label” or “orthogonal detectable moiety” as used herein refer to a detectable label (e.g., fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy5-Labeled Streptavidin, and R6G-Labeled Dibenzocyclooctyne.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent and the agent that cleaves each cleavable linker is different. In embodiments, an orthogonally is a cleavable linker that following cleavage the two separated entities (e.g., fluorescent dye, bioconjugate reactive group) do not further react and form a new orthogonally cleavable linker.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

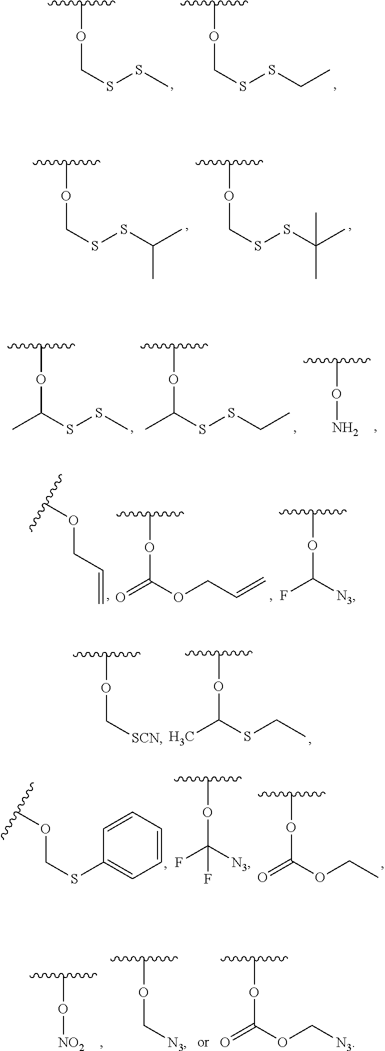

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label,” “detectable label,” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy®3). In embodiments, the cyanine moiety has 5 methine structures (i.e., cyanine 5 or Cy®5). In embodiments, the cyanine moiety has 7 methine structures (i.e., cyanine 7 or Cy®7).

The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

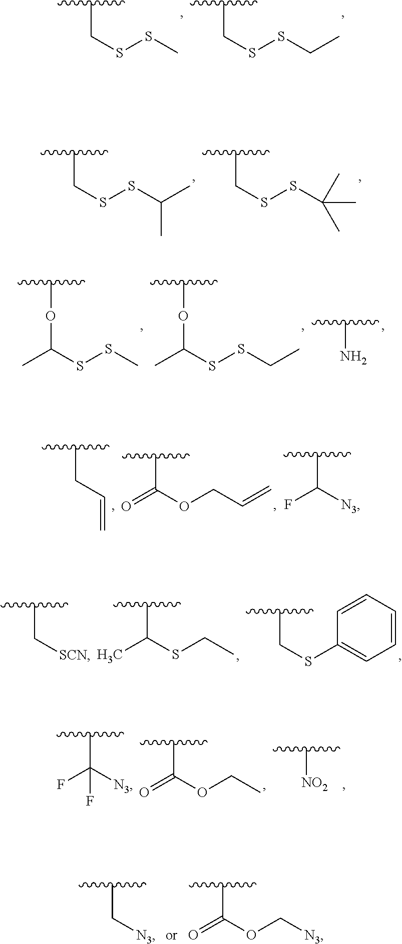

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

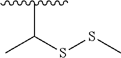 [SMILES: *C(C)SSC]

as described in U.S. Pat. No. 10,738,072, which is incorporated herein by reference for all purposes. For example, a nucleotide including a reversible terminator moiety may be represented by the formula:

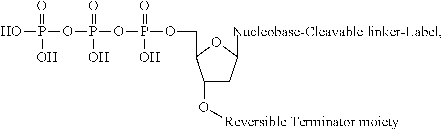

where the nucleobase is adenine or adenine analogue, thymine or thymine analogue, guanine or guanine analogue, or cytosine or cytosine analogue.

In some embodiments, a nucleic acid (e.g., a probe, such as a circularizable probe) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of barcodes is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide.

In embodiments, a nucleic acid (e.g., an padlock probe or circularizable probe) includes a sample barcode. In general, a “sample barcode” is a nucleotide sequence that is sufficiently different from other sample barcode to allow the identification of the sample based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides. As described herein, the barcode can be a nucleic acid that has a unique sequence, a unique fluorophore or a unique combination of fluorophores. A particular barcode can be unique relative to other barcodes. A “barcode” is a label, or identifier, that conveys or is capable of conveying information (e.g., information about an analyte in a sample). In embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In embodiments, barcodes may include a series of two or more segments or sub-barcodes (e.g., corresponding to “letters” or “code words” in a decoded barcode), each of which may comprise one or more of the subunits or building blocks used to synthesize the physical (e.g., nucleic acid) barcode molecules. For example, a nucleic acid barcode molecule may include two or more barcode segments, each of which includes one or more nucleotides. In embodiments, a barcode includes 1, 2, 3, 4, 5, 6, 7, 8, 9, 10, or more than 10 segments. In embodiments, each segment of a barcode molecule includes at least 1, 2, 3, 4, 5, 6, 7, 8, 9, 10, 12, 14, 16, 18, 20, or more than 20 subunits or building blocks. For example, each segment of a nucleic acid barcode molecule may include 1, 2, 3, 4, 5, 6, 7, 8, 9, 10, 12, 14, 16, 18, 20, or more than 20 nucleotides. In embodiments, two or more of the segments of a barcode may be separated by non-barcode segments, i.e., the segments of a barcode molecule need not be contiguous.

A “digital barcode” (or “digital barcode sequence”) is a representation of a corresponding physical barcode (or target analyte sequence) in a computer-readable, digital format as described above. A digital barcode may comprise one or more “letters” (e.g., 1, 2, 3, 4, 5, 6, 7, 8, 9, 10, 12, 14, 16, 18, 20, or more than 20 letters) or one or more “code words” (e.g., 1, 2, 3, 4, 5, 6, 7, 8, 9, 10, or more than 10 code words), where a “code word” comprises, e.g., 1, 2, 3, 4, 5, 6, 7, 8, 9, 10, 12, 14, 16, 18, 20, or more than 20 letters. In some instances, the sequence of letters or code words in a digital barcode sequence may correspond directly with the sequence of building blocks (e.g., nucleotides) in a physical barcode. In some instances, the sequence of letters or code words in a digital barcode sequence may not correspond directly with the sequence of building blocks in a physical barcode, but rather may comprise, e.g., arbitrary code words that each correspond to a segment of a physical barcode. For example, in some instances, the disclosed methods for decoding and error correction may be applied directly to detecting target analyte sequences (e.g., mRNA sequences) as opposed to detecting target barcodes, and the barcode probes used to detect the target analyte sequences may correspond to letters or code words that have been assigned to specific target analyte sequences but that do not directly correspond to the target analyte sequences.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. coli DNA polymerase I, Bacteriophage T4 DNA polymerase, Sequenase™ Taq DNA polymerase, DNA polymerase from Bacillus stearothermophilus, Bst 2.0 DNA polymerase, 9° N polymerase (exo-)A485L/Y409V, Phi29 DNA Polymerase ((φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol τ DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′—OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “ligase” refers to an enzyme that catalyzes the formation of a new phosphodiester bond as a result of joining the 5′-phosphoryl terminus of DNA or RNA to single-stranded 3′-hydroxyl terminus of DNA or RNA. Ligase enzymes can form circular DNA or RNA templates in a non-template driven reaction, and examples of ligase enzymes include, but are not limited to, as CircLigase™, Taq DNA Ligase, HiFi Taq DNA Ligase, T4 ligase, or Ampligase® DNA Ligase.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷ M, less than 10⁻⁸ M, less than 10⁻⁹ M, less than 10⁻¹⁰ M, less than 10⁻¹¹ M, or less than about 10⁻¹² M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor®, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure® XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction includes a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays including colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes. In embodiments, a cellular component is a biomolecule.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker).

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™ MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide. In some examples, cells in a tissue are configured in a mass and may not be free from one another. This disclosure describes methods of obtaining single biological samples (e.g., cells or nuclei) from tissues that can be used in various single biological samples (e.g., single-cell/nucleus) workflows. In some examples, blood cells (e.g., lymphocytes) can be considered a tissue. However, blood cells, like lymphocytes, generally are free from one another in the blood. The methods disclosed herein can be used to process those cells to obtain cells and/or nuclei, although dissociation steps may not be necessary when using those types of tissues. Generally, any type of tissue can be used in the methods described herein. Examples of tissues that may be used in the disclosed methods include, but are not limited to connective, epithelial, muscle and nervous tissue. In some examples, the tissues are from mammals. Tissues that contain any type of cells may be used. For example, tissues from abdomen, bladder, brain, esophagus, heart, intestine, kidney, liver, lung, lymph node, olfactory bulb, ovary, pancreas, skin, spleen, stomach, testicle, and the like. The tissue may be normal or tumor tissue (e.g., malignant). This example is not meant to be limiting. Although the conditions used in the disclosed may not be identical for different types of tissue, the methods may be applied to any tissue. The tissues used in the disclosed methods may be in various states. In some examples, the tissues used in the disclosed methods may be fresh, frozen, or fixed.

As used herein, the term “fresh,” generally in the context of a fresh tissue means that the tissue has recently been obtained from an organism, generally before any subsequent fixation steps, for example, flash freezing or chemical fixation. In embodiments, a fresh tissue is obtained from an organism about 1 second up to about 20 minutes before any fixation steps are performed. In embodiments, a fresh tissue is obtained from an organism about 1 second up to about 60 seconds before any fixation steps are performed. In embodiments, a fresh tissue is obtained from an organism about 30 seconds up to about 60 seconds before any fixation steps are performed. In embodiments, a fresh tissue is obtained from an organism about 1 minutes up to about 20 minutes before any fixation steps are performed. In embodiments, a fresh tissue is obtained from an organism about 1 minutes up to about 10 minutes before any fixation steps are performed. In embodiments, a fresh tissue is obtained from an organism about 1 minutes up to about 5 minutes before any fixation steps are performed. In embodiments, a fresh tissue is obtained from an organism about 30 seconds, about 1 minute, about 2 minutes, about 3 minutes, about 4 minutes, about 5 minutes, about 10 minutes, about 15 minutes, or about 20 minutes before any fixation steps are performed.

As used herein, the term “fix,” refers to formation of covalent bonds, such as crosslinks, between biomolecules or within molecules. The process of fixing tissue samples or biological samples (e.g., cells and nuclei) for example, is called “fixation.” The agent that causes fixation is generally referred to as a “fixative” or “fixing agent.” “Fixed biological samples” (e.g., fixed cells or nuclei) or “fixed tissues” refers to biological samples (e.g., cells or nuclei) or tissues that have been in contact with a fixative under conditions sufficient to allow or result in formation of intra- and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. In some examples, the tissue fixed is fresh tissue. In some examples, the tissue fixed may be frozen tissue. In some examples, the tissue fixed may not be dissociated. In some examples, the tissue fixed may be dissociated or partially dissociated (e.g., chopped, cut). In some examples, tissue that has been rapidly frozen and, perhaps, cut or chopped into pieces (e.g., small enough to fit into a tube or container used for fixation) may be used. In some examples, tissue may be dissociated or partially dissociated (e.g., cut, chopped) before or during fixation. In some examples, tissue that is fixed may not be dissociated. The frozen biological tissue can be fixed using a fixing agent, which is suitably an organic fixing agent. Suitable organic fixing agents include without limitation alcohols, ketones, aldehydes (e.g., glutaraldehyde), cross-linking agents, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis(sulfosuccinimidyl)suberate (BS3) and combinations thereof. A particularly suitable fixing agent is a formaldehyde-based fixing agent such as formalin, which is a mixture of formaldehyde and water. The formalin may include about 1% to about 15% by weight formaldehyde and about 85% to about 99% by weight water, suitable about 2% to about 8% by weight formaldehyde and about 92% to about 98% by weight water, or about 4% by weight formaldehyde and about 96% by weight water. In some examples, tissues may be fixed in 4% paraformaldehyde. Other suitable fixing agents will be appreciated by those of ordinary skill in the art (e.g., International PCT App. No. PCT/US2020/066705, which is incorporated herein by reference in its entirety).

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

As used herein, the term “substantially” refers to a condition in which a characteristic or property is exhibited to a total or near-total extent, recognizing that biological and chemical processes rarely, if ever, reach absolute completion. One of ordinary skill in the biological sciences will appreciate that reactions often involve inherent variability and may not achieve or avoid an absolute state. Accordingly, the term “substantially” accounts for the inherent limitations in biological and chemical systems, encompassing minor deviations that do not materially affect the intended outcome. For example, in the context of the phrase “wherein the reaction occurs at substantially the same temperature,” the term acknowledges that minor fluctuations in temperature may occur due to inherent system variability, yet the reaction remains within a range that does not significantly alter its efficiency or expected behavior.

As used herein, a “probe” (e.g., a first probe, a second probe, a third probe, etc.) refers to an agent that includes (1) a binding moiety capable of specifically binding to a biomolecule of interest and (2) a detectable label to enables the detection of the biomolecule of interest. The compositions and kits described herein utilize a probe to facilitate the serial detection of a biomolecule of interest in a cell or tissue by forming a complex including the probe described herein bound to the biomolecule described herein and detecting the complex. In embodiments, the probe further includes a quenching moiety attached to the probe via a cleavable linker. In embodiments, the probe is a specific binding reagent. In embodiments, the probe includes an antibody, single-chain Fv fragment (scFv), antibody fragment-antigen binding (Fab), affimer, or an aptamer. In embodiments, the probe is specific to a protein of interest. In embodiments, the probe is specific to an oligonucleotide of interest. In embodiments, the probe includes an antibody that is specific to a protein of interest. In embodiments, the probe includes an oligonucleotide that is specific to a nucleic acid of interest.

As used herein, a “complex” refers to a molecular entity formed by binding a probe described herein with a biomolecule described herein, wherein the complex includes a moiety that is capable of specifically binding to a biomolecule of interest (i.e., a biomolecule-specific binding agent) bound to the biomolecule. The compositions and kits described herein utilize a probe to facilitate the serial detection of a biomolecule of interest in a cell or tissue by forming a complex including the probe described herein bound to the biomolecule described herein and detecting the complex.

As used herein, a “specific binding reagent” refers to an agent that binds specifically to a particular biomolecule (e.g., carbohydrate, cell surface receptor, protein of interest, nucleic acid of interest, or lipid molecule of interest). Examples of a specific binding reagent include, but are not limited to, an antibody or target-specific oligonucleotide. As used herein, the term “protein-specific binding agent” refers to a molecule or agent that recognizes and binds to a protein or specific part of a protein with high affinity and specificity. Examples of a protein-specific protein binding agent include, but are not limited to, antibodies, aptamers, enzyme inhibitors, ligands for receptors, affinity tags, peptide-based protein binding agents, chelating agents for metalloproteins, and RNA interference agents. In embodiments, a protein-specific binding agent includes a protein-specific antibody conjugated to an oligonucleotide (referred herein as “protein-specific antibody-oligo (Ab-O) conjugates”), wherein the oligonucleotide in the protein-specific antibody-oligo is an oligonucleotide label as described herein.

A “monoclonal antibody” comprises a collection of identical molecules produced by a single B cell lymphocyte clone which are directed against a single antigenic determinant. Monoclonal antibodies can be distinguished from polyclonal antibodies in that monoclonal antibodies must be individually selected whereas polyclonal antibodies are selected in groups of more than one or, in other words, in bulk. Large amounts of monoclonal antibodies can be produced by immortalization of a polyclonal B cell population using hybridoma technology. Each immortalized B cell can divide, presumably indefinitely, and gives rise to a clonal population of cells that each expresses an identical antibody molecule. The individual immortalized B cell clones, the hybridomas, are segregated and cultured separately.

The term “polyclonal antibody” refers to an antibody that is produced from a different B cell lineages within the body. A polyclonal antibody is directed to many different antigenic determinants on the target cell surface and would bind with sufficient density to allow the effector mechanisms of the immune system to work efficiently.

An immunoglobulin (antibody) structural unit are typically tetrameric glycosylated proteins. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Thus, the term antibody, as used herein, includes antibody fragments either produced by the modification of whole antibodies, or those synthesized de novo either chemically or using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301. As appreciated by one of skill in the art, various antibody fragments can be obtained by a variety of methods, for example, digestion of an intact antibody with an enzyme, such as pepsin; or de novo synthesis.

The term “aptamer” refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their +complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

An “antigen” (Ag) is any substance that reacts specifically with antibodies or T lymphocytes (T cells). In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef, HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the biomolecule-specific binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype O11); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gplOO, dipeptidyl peptidase IV (DPPIV), adenosine deaminase-binding protein (ADAbp), cyclophilin b, colorectal associated antigen (CRC)-COI 7-1 A/GA733, carcinoembryonic antigen (CEA) and its immunogenic epitopes CAP-1 and CAP-2, etvβ, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulfites. These lists are not meant to be limiting

An “affimer” is a non-antibody protein that binds to target proteins with affinity in the nanomolar range. It behaves similarly to an antibody by binding tightly to its target molecule. Affimers are recombinant proteins that are typically engineered to mimic molecular recognition characteristics of monoclonal antibodies.

As used herein, the term “identification oligonucleotide” can also refer to a “barcode” or “index” or “unique molecular identifier (UMI)” and refers to a known nucleic acid sequence which has feature(s) that can be identified. Typically, an identification oligonucleotide is unique to a particular feature in a pool of identification oligonucleotide that differ from one another in sequence, and each of which is associated with a different feature. In embodiments, identification oligonucleotides are about or at least about 5, 6, 7, 8, 9, 10, 15, 20, 25, 30, 40, 50, 75 or more nucleotides in length. In embodiments, identification oligonucleotides are shorter than 20, 15, 10, 9, 8, 7, 6, or 5 nucleotides in length. In embodiments, identification oligonucleotides are 10-50 nucleotides in length, such as 15-40 or 20-30 nucleotides in length. In a pool of different identification oligonucleotides, identification oligonucleotides may have the same or different lengths. In general, identification oligonucleotides are of sufficient length and comprise sequences that are sufficiently different to allow the identification of associated features (e.g., a binding agent or analyte) based on identification oligonucleotides with which they are associated. In embodiments, an identification oligonucleotide can be identified accurately after the mutation, insertion, or deletion of one or more nucleotides in the identification oligonucleotide sequence, such as the mutation, insertion, or deletion of 1, 2, 3, 4, 5, or more nucleotides. In embodiments, each identification oligonucleotide in a plurality of identification oligonucleotides differs from every other identification oligonucleotide in the plurality by at least three nucleotide positions, such as at least 3, 4, 5, 6, 7, 8, 9, 10, or more nucleotide positions.

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent.

The terms “fluorophore,” “fluorescent agent,” “fluorescent dye,” or “fluorescent dye moiety” are used interchangeably and refer to a substance, compound, agent, or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon Green®, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5™, DRAQ7™, or CyTRAK Orange™), pyrene derivatives (e.g., cascade blue and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF® dye, DRAQ™, CyTRAK™, BODIPY®, Alexa Fluor®, DyLight®, Atto®, Tracy, FluoProbes®, Abberior® Dyes, DY dyes, MegaStokes Dyes, Sulfo Cy®, Seta dyes, SeTau dyes, Square Dyes, Quasar® dyes, Cal Fluor™ dyes, SureLight® Dyes, PerCP, Phycobilisomes, APC, APCXL™ RPE, and/or BPE. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

The term “rhodamine” as is used in accordance with its ordinary meaning in the art and refers to a detectable moiety including a xanthene backbone. Structurally, rhodamine is a family of related polycyclic dyes with a xanthene core, i.e.,

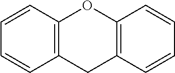 [SMILES: c1ccc2c(c1)Cc1ccccc1O2]

(xanthene). Generally speaking, functional groups on the conjugated moiety of the xanthene core have the ability to fine tune the fluorescent colors. Non-limiting examples of rhodamine dyes include Rhodamine B, Rhodamine 6G, Rhodamine 123, and Rhodamine WH. Rhodamine derivatives have also been disclosed, such as in PCT Int. Appl, WO 2009108905; U.S. Pat. Nos. 5,728,529; 5,686,261; and by Kim et al. (Journal of Physical Chemistry A (2006), 110(1), 20-27)).

The term “sulforhodamine 101 moiety” is used in accordance with its ordinary meaning in the art and refers to detectable moiety containing xanthene backbone with the following structure:

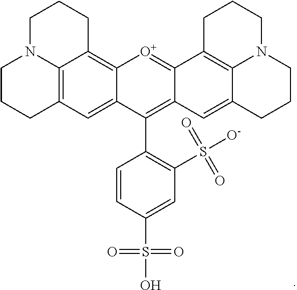 [SMILES: O=S(=O)([O-])c1cc(S(=O)(=O)O)ccc1-c1c2cc3c4c(c2[o+]c2c5c6c(cc12)CCCN6CCC5)CCCN4CCC3]

The sulforhodamine 101 moiety is a red fluorescent dye and commonly used for astrocyte identification. An example of a commercially available dye with a sulforhodamine 101 moiety is Texas Red.

The term “fluorescein moiety” is used in accordance with its ordinary meaning in the art and refers to a detectable moiety containing xanthene backbone having the following structural formula:

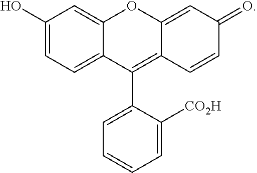 [SMILES: O=C(O)c1ccccc1-c1c2ccc(=O)cc-2oc2cc(O)ccc12]

Dyes with fluorescein moieties are commonly used as fluorescent probes in life sciences and medical applications due to their hydrophilicity, high absorptivity, and high quantum yield. Examples of detectable agents containing fluorescein moieties include fluorescein reactive dyes, which are fluorescein dyes derivatized with different bioconjugation moieties (e.g., maleimide, NHS, or isothiocyanate moieties).

The term “fluorescein isothiocyanate moiety” is used in accordance with its ordinary meaning in the art and refers to a detectable moiety containing xanthene backbone derived from a fluorescein moiety. Detectable agents harboring a fluorescein isothiocyanate moiety has the following structural formula:

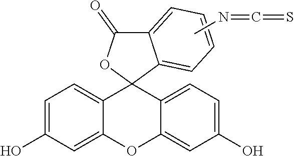

and are primarily used to label primary amines of a biomolecule. Commercially available forms of detectable agents with a fluorescein isothiocyanate moiety include fluorescein 5-isothiocyanate (5-FITC), fluorescein 6-isothiocyanate (6-FITC), or a mixture of the two isomers.

The term “cyanine” or “cyanine moiety” is used in accordance with its ordinary meaning in the art and refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy®5). In embodiments, the cyanine moiety has 7 methine structures (i.e., cyanine 7 or Cy®7). Cyanine dyes refer to a family of dyes in which the chromophoric system includes conjugated double bonds connecting two end groups consisting of an electron acceptor and an electron donor. There are three types of cyanine dyes: (1) closed chain cyanines of the general structure:

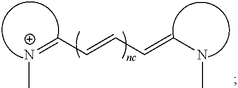

(2) hemicyanines of the general structure:

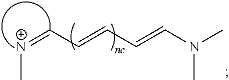

and (3) open chain cyanines of the general structure:

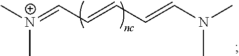

where nc is an integer from 1 to 9.

The term “indocyanine green moiety” is used in accordance with its ordinary meaning in the art and refers to a detectable moiety from the cyanine family of dyes. Specifically, an indocyanine green moiety consists of a cyanine 7 dye moiety of the following structure:

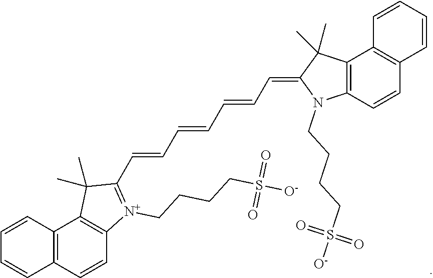 [SMILES: CC1(C)C(/C=C/C=C/C=C/C=C2\N(CCCCS(=O)(=O)[O-])c3ccc4ccccc4c3C2(C)C)=[N+](CCCCS(=O)(=O)[O-])c2ccc3ccccc3c21]

The term “triarylmethane” is used in accordance with its ordinary meaning in the art and refers to a detectable moiety containing a triarylmethane backbone. A triarylmethane dye is derived from a triaryl methane compound and is used for colorimetric assays, analytical chemistry, and are used to color fabrics and plastics, as well as in inks and paints. Examples of triaryl methane dyes include Malachite Green, Crystal Violet, Methyl Violet, Methylene Blue, and Phenol Red.

The term “coumarin moiety” used in accordance with its ordinary meaning in the art and refers to a detectable moiety containing a benzene and a-pyrone rings of the general structure:

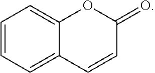 [SMILES: O=c1ccc2ccccc2o1]

Dyes with a coumarin moiety are typically excited with electromagnetic radiation from the UV range and emit between 400-470 nm. Examples of commercially available dyes derived containing a coumarin moiety include DiFMUP (6,8-difluoro-4-methylumbelliferyl phosphate) and AMC (7-amino-4-methylcoumarin).

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO™ Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. In embodiments, the tissue is in a channel of a flow cell.

As used herein, the term “gasket” refers to an element that separates the first solid support and the second solid support to define a reaction chamber on the second solid support, wherein the reaction chamber includes a defined gap or channel through which liquid can flow or be contained. In embodiments, a gasket is a spacer element. In embodiments, the thickness (also referred herein as the “depth” or “height” of the channel) may be altered by modulating the height of the gasket or spacer element. In embodiments, the gasket or spacer element includes a peel-off backing designed to form a sealed reaction chamber on the second solid support when adhered to the first solid support. This design ensures the creation of defined channels necessary for fluid flow and biochemical reactions within the assembled flow cell (e.g., flow cell assembly described herein). An example of a gasket or spacer element includes, but is not limited to, those used in the NovaSeq™6000 S4 flow cells, commercialized by Illumina®, which is depicted in Poovathingal et al. (doi: 10.1101/2024.02.22.581576).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions, Systems, & Kits

In an aspect is provided a composition including a first sequencing primer and a second sequencing primer, wherein the first sequencing primer includes one or more cleavable sites. In embodiments, the cleavable site includes a disulfide bond, a deoxyuracil triphosphate (dUTP), a deoxy-8-oxo-guanine triphosphate (d-8-oxoG), a methylated nucleotide, or a ribonucleotide.

In embodiments, the composition includes a specific binding reagent (alternatively referred to herein as a specific binding agent) covalently attached to an oligonucleotide, and a circularizable probe configured to bind to the oligonucleotide.

In embodiments, the specific binding agent is a monoclonal antibody or a polyclonal antibody. In embodiments, the specific binding agent is capable of binding (e.g., capable of specifically binding) to an actin filament of a cell, a plasma membrane of a cell, a mitochondria of a cell, the endoplasmic reticulum of a cell, a tubule of the endoplasmic reticulum, a cisternae of the endoplasmic reticulum, sheets and tubules of the endoplasmic reticulum, a nuclear envelope of the endoplasmic reticulum, a Golgi apparatus of a cell, cisternae of the Golgi apparatus, a lysosome of a cell, phosphatidylserine, a cell surface carbohydrate, or a transferrin receptor. In embodiments, the specific binding agent is capable of binding a carbohydrate on a cell surface. In embodiments, the specific binding agent is capable of binding a glycolipid, a glycoprotein, an a-glucopyranosyl residue on a cell membrane, an N-acetylglucosaminyl residue on a cell membrane, an N-acetylneuraminic acid (sialic acid) on a cell membrane, peroxisome, a nucleus, an endosome, or a cytoskeletal protein. In embodiments, the cytoskeletal protein includes talin. In embodiments, the cytoskeletal protein includes tubulin. In embodiments, the specific binding agent is a monovalent phalloidin molecule, monovalent wheat germ agglutinin molecule, monovalent concanavalin A molecule, an annexin molecule, transferrin molecule, lectin molecule, or Hoescht 33342. In embodiments, the specific binding agent is a cell paint (see, e.g., Gustafsdottir S. M. et al. PLoS One. 2013 Dec. 2; 8(12):e80999).

In embodiments, the specific binding agent is an antibody, single-chain Fv fragment (scFv), antibody fragment-antigen binding (Fab), affimer, or an aptamer. In embodiments, the specific binding reagent is an antibody. In embodiments, the specific binding reagent is a single-chain Fv fragment (scFv). In embodiments, the specific binding reagent is an antibody fragment-antigen binding (Fab). In embodiments, the specific binding reagent is an affimer. In embodiments, the specific binding reagent is an aptamer. In embodiments, the specific binding agent is specific for a target molecule described herein. In embodiments, the specific binding agent include on average between 1.0 and 5, or between 1 and 2.5 oligonucleotides conjugated to the specific binding agent. In embodiments, the specific binding agent includes, on average, one oligonucleotide conjugated to the specific binding agent. In embodiments, the specific binding agent includes, on average, two oligonucleotides conjugated to the specific binding agent. In embodiments, the specific binding agent includes, on average, three oligonucleotides conjugated to the specific binding agent. In embodiments, the specific binding agent includes, on average, four oligonucleotides conjugated to the specific binding agent. In embodiments, the specific binding agent includes, on average, five oligonucleotides conjugated to the specific binding agent.

The stoichiometry of the conjugation reaction to form the antibody-oligonucleotide conjugates, for example, the antibody-oligonucleotide conjugates, may include one equivalent of antibody (e.g., a modified antibody to include a bioconjugate reactive moiety) and at least 0.5 equivalents of modified oligonucleotide (e.g., modified to include a bioconjugate reactive moiety, such that upon reacting with the bioconjugate reactive moiety on the antibody a bioconjugate linkage is formed). Other examples are at least 1.0 equivalent, at least 1.5 equivalents, at least 2.0 equivalents, at least 2.5 equivalents, at least 3.0 equivalents, at least 3.5 equivalents, or at least 4.0 equivalents of oligonucleotide. The stoichiometry of the conjugation reaction to form the conjugates, may include one equivalent of antibody (e.g., a modified antibody to include a bioconjugate reactive moiety) and between about 0.5 and about 2.0 of modified oligonucleotide, for example, between about 1.5 and about 2.5 equivalents, between about 2.0 and about 2.5 equivalents, between about 2.0 and about 3.0 equivalents, between about 2.5 and about 3.5 equivalents, between about 3.0 and about 3.5 equivalents, between about 3.0 and about 4.0 equivalents, or between about 3.5 and about 4.5 equivalents modified oligonucleotide. In embodiments, the stoichiometry of the conjugation reaction may be adjusted to form antibody-oligonucleotide conjugates that retain sufficient immunoreactivity of the antibody. A suitable modified oligonucleotide may be prepared by incorporating amino groups either 3′,5′ or internally using other methods and reagents. For example, the modified oligonucleotide may be prepared by reacting with a moiety that is a bifunctional molecular reagent, such as an aromatic aldehyde or ketone, aromatic hydrazino or oxyamino modification reagent, to incorporate a hydrazino or oxyamino function respectively. For example, the modified oligonucleotide may be prepared by reacting with a bifunctional molecular reagent containing a first reactive component that forms a covalent bond with the oligonucleotide, and a second reactive component that may form a linkage with a complementary reactive component on a modified antibody (e.g., an antibody containing a bioconjugate reactive moiety). In embodiments, the second reactive component may be protected such that it will not react until removed following incorporation onto the oligonucleotide. In embodiments, the bioconjugate reactive moiety is HyNic (6-HydrazinoNicotinamide). In embodiments, the modified antibody includes a HyNic-modified biomolecule (i.e., covalently modified to display a hydrazinonicotinate reactive moiety). The modified oligonucleotide may also include a 4-FB-modified oligonucleotide (i.e., covalently modified to display a 4-formylbenzamide moiety).

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

Specific binding agents such as the Ab-O conjugates, as described herein, individually consist of a biomolecule-binding domain with specificity to the target analyte or biomolecule, and a nucleic acid domain linked, coupled, or conjugated thereto. For example, the biomolecule-binding domain can be for example a nucleic acid “aptamer” (Fredriksson et al (2002) Nat Biotech 20:473-477) or can be proteinaceous, such as a monoclonal or polyclonal antibody (Gullberg et al (2004) PNAS USA 101:8420-8424). A suitable antibody or immunoglobulin may include, for example, natural antibodies, artificial antibodies, genetically engineered antibodies, monovalent antibodies, polyvalent antibodies, monoclonal antibodies, polyclonal antibodies, camelids, monobodies, scFvs and/or fragments or derivatives thereof. In embodiments, the antibody or immunoglobulin molecules may be monoclonal, polyclonal, monospecific, polyspecific, humanized, single-chain, chimeric, camelid single domain, shark single domain, synthetic, recombinant, hybrid, mutated, CDR-grafted antibodies, and/or fragments or derivatives thereof. In embodiments, antibodies may be derived from mammal species, for example, rat, mouse, goat, guinea pig, donkey, rabbit, horse, lama, camel, or avian species, such as chicken or duck. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species. Affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site. The antibody may be chemically modified to include a hapten, for example a small molecule or a peptide. The hapten may be a nitrophenyl group, a dinitrophenyl group, a digoxygenin, a biotin, a Myc tag, a FLAG tag, an HA tag, an S tag, a Streptag, a His tag, a V5 tag, a ReAsh tag, a FlAsh tag, a biotinylation tag, or Sfp tag.

In embodiments, the oligonucleotide is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In embodiments, the oligonucleotide is 20 to 30 nucleotides. In embodiments, the oligonucleotide is 30 to 40 nucleotides. In embodiments, the oligonucleotide is 40 to 50 nucleotides.

In embodiments, the oligonucleotide is attached to a specific binding agent (e.g., an antibody) via a linker (e.g., a bioconjugate linker). In embodiments, the oligonucleotide is attached to the protein-specific binding agent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety. In embodiments, the oligonucleotide includes a barcode, wherein the barcode is a known sequence associated with the specific binding agent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

In embodiments, the circularizable polynucleotide is referred to as a padlock probe. In embodiments, the padlock probe includes a primer binding sequence from a known set of primer binding sequences. In embodiments, the circularizable polynucleotide includes only one primer binding sequence, wherein the primer binding sequence serves as the amplification primer binding sequence and sequencing primer binding sequence. In embodiments, the circularizable polynucleotide includes at least two primer binding sequences from a known set of primer binding sequences. In embodiments, the circularizable polynucleotide includes two or more primer binding sequences from a known set of primer binding sequences. In embodiments, a plurality of circularizable polynucleotides may include up to 20 different primer binding sequences from a known set of primer binding sequences. In embodiments, the plurality of circularizable polynucleotides includes up to 10 different primer binding sequences from a known set of primer binding sequences. In embodiments, the plurality of circularizable polynucleotides includes up to 5 different primer binding sequences from a known set of primer binding sequences. In embodiments, the circularizable polynucleotide includes two or more sequencing primer binding sequences from a known set of sequencing primer binding sequences. In embodiments, the circularizable polynucleotide includes 1, 2, 3, 4, 5, 6, 7, 8, 9, or 10 primer binding sequences from a known set of primer binding sequences. In embodiments, the circularizable polynucleotide includes two or more different primer binding sequences from a known set of primer binding sequences. In embodiments, the circularizable polynucleotide includes 1, 2, 3, 4, 5, 6, 7, 8, 9, or 10 different primer binding sequences from a known set of primer binding sequences. In embodiments, the circularizable polynucleotide includes 2 to 5 primer binding sequences from a known set of primer binding sequences. In embodiments, the circularizable polynucleotide includes 2 to 5 different primer binding sequences from a known set of primer binding sequences. In embodiments, the circularizable polynucleotide includes 2 to 5 sequencing primer binding sequences from a known set of sequencing primer binding sequences. In embodiments, the circularizable polynucleotide includes 2 to 5 different sequencing primer binding sequences from a known set of sequencing primer binding sequences.

In embodiments, the circularizable polynucleotide includes about 50 to about 150 nucleotides. In embodiments, the circularizable polynucleotide includes about 70 to about 130 nucleotides. In embodiments, the circularizable polynucleotide includes about 50 to about 300 nucleotides. In embodiments, the circularizable polynucleotide includes about 50 to about 500 nucleotides. In embodiments, the circularizable polynucleotide includes about or more than about 50, 75, 100, 125, 150, 175, 200, 250, 300, 350, 400, or 500 nucleotides. In embodiments, the circularizable polynucleotide includes less than about 50, 75, 100, 125, 150, 175, 200, 250, 300, 350, 400, or 500 nucleotides. In embodiments, the circularizable polynucleotide (i.e., circularizable polynucleotide probe) includes about 70, 75, 80, 85, 90, 95, 100, 105, 110, 115, 120, 125, 130, 135, 140, 145, or 150 nucleotides.

In embodiments, the circularizable polynucleotide includes at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the circularizable polynucleotide includes a barcode sequence. In embodiments, the circularizable polynucleotide includes a barcode nucleotide. In embodiments, the barcode sequence is at least 1, 2, 3, 4, 5, 6, 7, 8, 9, 10, 11, 12, 13, 14, or 15 nucleotides in length. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a circularizable polynucleotide described herein or circularizable polynucleotide probe described herein that is conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest.

In embodiments, the barcode sequence is selected from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the circularizable polynucleotide includes a barcode nucleotide. A barcode nucleotide refers to a single nucleotide which may serve as a differentiating feature among targets. Detecting four different targets using a single nucleotide as a barcode may involve the use of a common primer and the incorporation of differently colored labeled nucleotides into the primer, rendering simultaneous detection of multiple targets. For example, one may bind a common primer to each of the four separate targets (e.g., amplification products arising from four separate target molecules). This common primer is designed to hybridize to a specific region shared among the targets, serving as a starting point for the subsequent incorporation of nucleotides. With a polymerase, differently colored labeled nucleotides are incorporated into the newly synthesized DNA strand opposite the barcode nucleotide. Each of the four types of nucleotides (adenine, thymine, cytosine, and guanine) is tagged with a unique fluorescent dye, with each dye emitting a distinct color upon excitation. For instance, adenine might be tagged with a green dye, thymine with blue, cytosine with red, and guanine with yellow. As the primer is extended, a colored nucleotide is incorporated to a position complementary to the barcode nucleotide. Detection is then based on the color emitted upon fluorescence excitation. For example, if the barcode nucleotide is adenine, then the complementary thymine, labeled with a blue fluorophore, is incorporated into the extending strand. The presence of the target adenine is then identified by the emission of a blue fluorescence signal. This color-coded system allows for the distinct identification of each of the four targets based on the specific fluorescence emitted by the incorporated nucleotides.

In an aspect is provided a cell, wherein the cell includes the composition as described herein. In another aspect is a tissue, wherein the tissue includes the composition as described herein. In another aspect is provided a target molecule, wherein the target molecule is bound to a composition as described herein. In embodiments, the target molecule is PD-L1, CD8, CD3, PD-1, CD45, CD4, CD68, CD11c, FoxP3, α-SMA, CD20, Ki67, CD56, CD31, CTLA-4/CD152, CTLA-4/CD153, p53, PanCK, CD45RO, CD45RA, and/or HLA-DR. In embodiments, the target molecule is selected from the group: PD-L1, CD8, CD3, PD-1, CD45, CD4, CD68, CD11c, FoxP3, α-SMA, CD20, Ki67, CD56, CD31, CTLA-4, PanCK, CD45RO, CD45RA, ATPase, Pan-Cadherin, Vimentin, Beta-2-microglobulin, and HLA-DR.

In an aspect is provided a cell or tissue including an oligonucleotide described herein, wherein the oligonucleotide is covalently attached to a specific binding agent that is an antibody, single-chain Fv fragment (scFv), antibody fragment-antigen binding (Fab), affimer, aptamer, enzyme, peptide, Molecular Imprinted Polymer (MIP), DARPin (Designed Ankyrin Repeat Protein), peptoid, or lectin. In embodiments, the cell or tissue further includes a first blocking oligonucleotide hybridized to a first sequence of the oligonucleotide described herein, and a second blocking oligonucleotide hybridized to a second sequence of the oligonucleotide described herein. In embodiments, the cell further includes a polynucleotide probe including a first binding sequence and a second binding sequence hybridized to the oligonucleotide described herein.

In embodiments, the target molecule is PD-L1. PD-L1 may be highly expressed in a variety of malignancies, for example lung cancer. Detecting PD-L1 may be useful in determining the effectiveness of gene therapy or systemic immunotherapy in blocking the PD-1 and PD-L1 checkpoints. In embodiments, detecting PD-L1 in TNBC cells or tissues is useful for evaluating patients for immunotherapy.

In embodiments, the target molecule is CD8 (e.g., CD8A and/or CD8B). In embodiments, detecting CD8 is useful for determining cytotoxic T cell infiltration into a cell or tissue.

In embodiments, the target molecule is CD3. In embodiments, the target molecule is CD3E (CD3 epsilon). CD3 is typically bound to the membranes of all mature T-cells.

In embodiments, the target molecule is PD1. PD1 often shows high and sustained expression levels during persistent antigen encounter, and is expressed by conventional T cells, regulatory T cells, B cells, and natural killer cells. In embodiments, detecting PD1 is useful as an immune checkpoint protein. In embodiments, Programmed cell death 1 receptor (PD-1) and its ligand programmed cell death ligand 1 (PD-L1) are immune checkpoint proteins found on the cell surface of T cells. Under physiological conditions their interaction results in T cell immune suppression, but diseases (e.g., cancer) disrupt normal function to escape immune detection.

In embodiments, the target molecule is CD4. CD4 is expressed in most neoplasms derived from T helper cells. For example, it is possible to use CD4 detection on tissue samples to identify most forms of peripheral T cell lymphoma and related malignant conditions. CD4 is also associated with a number of autoimmune diseases such as vitiligo and type I diabetes mellitus.

In embodiments, the target molecule is CD68. CD68 is typically found in the cytoplasmic granules of a range of different blood cells and myocytes, and is useful as a diagnostic marker for cells of the macrophage lineage, including monocytes, histiocytes, giant cells, Kupffer cells, and osteoclasts. Due to the presence of CD68 in macrophages, detecting CD68 is useful in diagnosing conditions related to proliferation or abnormality of these cells, such as malignant histiocytosis, histiocytic lymphoma, and Gaucher's disease. CD68 alone or in combination with other cell markers of tumor-associated macrophages is correlated with positive predictive value as a prognostic marker of survival in cancer patients.

In embodiments, the target molecule is CD11c. CD11c is a marker for dendritic cells, which are bone-marrow-derived cells found in the blood, epithelial and lymphoid tissues. CD11c is an approximately 150 kDa type I transmembrane glycoprotein protein and part of the complement receptor 4 (CR4). Together with CR3, named also Mac-1 or CD11b/CD18, CR4 belongs to the β2 integrin family of adhesion molecules. CD11c is not restricted to dendritic cells, but also expressed by other immune cells, such as macrophages in the alveoli and adipose tissues.

In embodiments, the target molecule is FoxP3. Alterations in numbers of regulatory T cells and in particular those that express Foxp3 are found in a number of disease states. For example, down-regulation of Foxp3 expression has been reported in tumor samples derived from breast, prostate, and ovarian cancer patients. In embodiments, detection of human FoxP3 is associated with regulatory T cell detection.

In embodiments, the target molecule is ACTA2 (commonly referred to as alpha-smooth muscle actin or α-SMA) is used as a marker of myofibroblast formation.

In embodiments, the target molecule is CD20. CD20 is expressed on all stages of B cell development from pre-B cells in the bone-marrow through immature, naive, mature and memory cells in lymphoid tissues and blood, though the expression is lost in terminally differentiated plasma cells. CD20 is a transmembrane protein consisting of four hydrophobic transmembrane domains, one intracellular domain and two extracellular loops. In embodiments, the detection of CD20 is associated with B cell malignancies, such as B-cell lymphomas, hairy cell leukemia, B-cell chronic lymphocytic leukemia, and melanoma cancer stem cells. Due to CD20 remains present on the cells of most B-cell neoplasms, and is absent on otherwise similar appearing T-cell neoplasms, detecting CD20 can be useful in diagnosing conditions such as B-cell lymphomas and leukemias.

In embodiments, the target molecule is Ki-67. Ki-67 is a nuclear protein that is associated with cellular proliferation and ribosomal RNA transcription.

In embodiments, the target molecule is CD56. Normal cells that stain positively for CD56 include natural killer cells, activated T cells, the brain and cerebellum, and neuroendocrine tissues. Additional tumor tissue sections that are CD56 positive include CD56-positive include tumor tissue section associated with myeloma, myeloid leukemia, neuroendocrine tumors, Wilms' tumor, neuroblastoma, extranodal NK/T-cell lymphoma, nasal type, pancreatic acinar cell carcinoma, pheochromocytoma, paraganglioma, and small cell lung carcinoma.

In embodiments, the target molecule is CD31. CD31 is used primarily to demonstrate the presence of endothelial cells in tissue sections. CD31 is typically found on the surface of platelets, monocytes, neutrophils, and some types of T-cells, and endothelial cell intercellular junctions. Expression of CD31 is correlated hemangioma, angiosarcoma, Kaposi's sarcoma, breast carcinoma, glioblastoma, colon carcinoma, and skin carcinoma.

In embodiments, the target molecule is CTLA-4. CTLA-4 is expressed in regulatory T cells and typically upregulated in conventional T cells after activation.

In embodiments, the target molecule is PanCK.

In embodiments, the target molecule is HLA-DR (alternatively referred to as HLA-DRA). HLA-DR is expressed on the surface of various antigen presenting cells such as B lymphocytes, dendritic cells, and monocytes/macrophages, and plays a role in the immune system and response by presenting peptides derived from extracellular proteins, in particular, pathogen-derived peptides to T cells.

In embodiments, the target molecule is vimentin. Vimentin is a type III intermediate filament (IF) protein and is a cytoskeletal component of mesenchymal cells. Vimentin is used as a marker of mesenchymally-derived cells or cells undergoing an epithelial-to-mesenchymal transition (EMT). Vimentin is useful at ascertaining the cell shape.

In embodiments, the target molecule is CD45. CD45 is an antigen expressed on immunological and hematological systems but is not expressed on red blood cells and platelets. Aberrant expression of CD45 is associated with leukemia and lymphoma. Expression of the six isoforms of CD45 contributes to lymphocyte survival, cytokine response, and T-cell receptor signaling.

In embodiments, the target molecule is CD45RO. CD45RO is an isoform of CD45 and is expressed by human memory T cells and activated T lymphocytes. In embodiments, the target molecule is CD45RA. CD45RA is an isoform of CD45 and is expressed in a subset of T cells, B cells, and monocytes.

In embodiments, the target molecule is ATPase. ATPase represents a superfamily involved in the synthesis of ATP from the phosphorylation of ADP, which is coupled by the transport of proton ions across membranes.

In embodiments, the target molecule is pan-cadherin. Pan-cadherin is a member of the cadherin protein family, and is a cell-cell adhesion glycoprotein that is critical for tissue architecture, cell differentiation, cell shape, cell polarity, growth and migration, particularly in epithelial tissues.

In embodiments, the target molecule is beta 2-microglobulin (B2M). The expression of this marker indicates the activation of the immune system. Aberrant expression of B2M is also observed in hematologic and renal disorders.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the cell is a cancer cell. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface.

In embodiments, the methods are performed in situ on isolated cells or in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or both proteins and lipids.

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the substrate is an alkaline earth boro-aluminosilicate glass substrate. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the solid support or substrate described herein includes one or more channels. In embodiments, the solid support or substrate includes a channel bored into solid support or substrate. In embodiments, the solid support or substrate includes a plurality of channels solid support or substrate. In embodiments, the solid support or substrate includes 2, 3, or 4 channels bored into solid support or substrate. In embodiments, the width of the channel is from about 1 to 5 mm, 5 mm to 10 mm, or 10 mm to 15 mm. In embodiments, the channel is a reaction chamber on the solid support or substrate. In embodiments, the cell or tissue is immobilized in a channel bored onto the solid support or substrate.

In an aspect is provided a solid support (e.g., a planar solid support) including a tissue section and plurality of particles as described herein, including embodiments. In embodiments, the tissue section is embedded in an embedding material, for example an embedding material including paraffin wax, polyepoxide polymer, polyacrylic polymer, agar, gelatin, celloidin, cryogel, optimal cutting temperature (OCT) composition, glycols, or a combination thereof. In embodiments, the tissue section includes a thickness of about 1 μm to about 20 μm. In embodiments, the tissue includes a thickness of about 1 μm to about 10 μm. In embodiments, the tissue includes a thickness of about 2 μm to about 3 μm. In embodiments, the tissue includes a thickness of about 4 μm to about 6 μm. In embodiments, the tissue includes a thickness of about 4 μm. In embodiments, the tissue includes a thickness of about 5 μm. In embodiments, the tissue includes a thickness of about 6 μm. In embodiments, the tissue includes a thickness of about 7 μm. In embodiments, the tissue includes a thickness of about 8 μm. In embodiments, the tissue includes a thickness of about 9 μm. In embodiments, the tissue includes a thickness of about 10 μm.

In embodiments, the tissue section includes a tissue or a cell (e.g., a plurality of cells such as blood cells). In embodiments, the tissue section includes one or more cells. In embodiments, the tissue section is embedded in an embedding material including paraffin wax, polyepoxide polymer, polyacrylic polymer, agar, gelatin, celloidin, cryogel, optimal cutting temperature (OCT) compositions, glycols, or a combination thereof. In embodiments, the tissue section is embedded in an embedding material including paraffin wax. In embodiments, the tissue section is embedded in an embedding material including a polyepoxide polymer. In embodiments, the tissue section is embedded in an embedding material including polyacrylic polymer. In embodiments, the tissue section is embedded in an embedding material including agar. In embodiments, the tissue section is embedded in an embedding material including gelatin. In embodiments, the tissue section is embedded in an embedding material including celloidin. In embodiments, the tissue section is embedded in an embedding material including a cryogel. In embodiments, the tissue section is embedded in an embedding material including an optimal cutting temperature (OCT) composition. In embodiments, the tissue section is embedded in an embedding material including one or more glycols. Tissue sections may be obtained from a subject by any means known and available in the art. In embodiments, a tissue section, e.g., a tumor tissue sample, is obtained from a subject by fine needle aspiration, core needle biopsy, stereotactic core needle biopsy, vacuum-assisted core biopsy, or surgical biopsy. In embodiments, the surgical biopsy is an incisional biopsy, which removes only part of the suspicious area.

In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm. In embodiments, the thickness of the tissue section is about 1 μm. In embodiments, the thickness of the tissue section is about 2 μm. In embodiments, the thickness of the tissue section is about 3 μm. In embodiments, the thickness of the tissue section is about 4 μm. In embodiments, the thickness of the tissue section is about 5 μm. In embodiments, the thickness of the tissue section is about 6 μm. In embodiments, the thickness of the tissue section is about 7 μm. In embodiments, the thickness of the tissue section is about 8 μm. In embodiments, the thickness of the tissue section is about 9 μm. In embodiments, the thickness of the tissue section is about 10 μm. In embodiments, the thickness of the tissue section is about 11 μm. In embodiments, the thickness of the tissue section is about 12 μm. In embodiments, the thickness of the tissue section is about 13 μm. In embodiments, the thickness of the tissue section is about 14 μm. In embodiments, the thickness of the tissue section is about 15 μm. In embodiments, the thickness of the tissue section is less than about 10 μm. In embodiments, the thickness of the tissue section is less about 6 μm, 7 μm, 8 μm, 9 μm or 10 μm. In embodiments, the cell or tissue is obtained via Laser capture microdissection (LCM). LCM is a method for isolating in micrometer-scale tissue or even single cells while retaining spatial information to link histology with molecular measurements. In LCM, a region of interest in the tissue section is isolated through laser cutting.

In embodiments, the tissue section includes a tissue or a cell. Biological tissue samples suitable for use with the methods and systems described herein generally include any type of tissue samples collected from living or dead subjects, such as, for example, tumor tissue and autopsy samples. Tissue samples may be collected and processed using the methods and systems described herein and subjected to microscopic analysis immediately following processing, or may be preserved and subjected to microscopic analysis at a future time, e.g., after storage for an extended period of time. In some embodiments, the methods described herein may be used to preserve tissue samples in a stable, accessible and fully intact form for future analysis. For example, tissue samples, such as, e.g., human tumor tissue samples, may be processed as described herein and cleared to remove a plurality of cellular components, such as, e.g., lipids, and then stored for future analysis. In some embodiments, the methods and systems described herein may be used to analyze a fresh tissue section. In some embodiments, the methods and systems described herein may be used to analyze a previously-preserved (e.g., previously fixed) or stored tissue section (e.g., tissue sample). For example, in some embodiments a previously-preserved tissue sample that has not been subjected to a sample preparation process described herein may be processed and analyzed as described herein. In particular methods, a tissue sample is frozen prior to being processed as described herein.

In embodiments, tissue sections are tumor tissue samples. Tumor samples may contain only tumor cells, or they may contain both tumor cells and non-tumor cells. In particular embodiments, a tissue section includes only non-tumor cells. In particular embodiments, the tumor is a solid tumor. In particular embodiments, the tissue section is obtained from or includes an adrenal cortical cancer, anal cancer, aplastic anemia, bile duct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal or squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment, is a tissue section obtained from a subject diagnosed with or suspected of having any of these tumors or cancers.

The methods of the invention can be used to characterize a cancer or metastasis thereof, including without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryoides, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer under analysis may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, brain cancer, kidney cancer, ovarian cancer, stomach cancer, skin cancer, bone cancer, gastric cancer, pancreatic cancer, glioma, glioblastoma, hepatocellular carcinoma, papillary renal carcinoma, head and neck squamous cell carcinoma, leukemia, lymphoma, myeloma, or a solid tumor.

Tissue sections may be obtained from a subject by any means known and available in the art. In particular embodiments, a tissue section, e.g., a tumor tissue sample, is obtained from a subject by fine needle aspiration, core needle biopsy, stereotactic core needle biopsy, vacuum-assisted core biopsy, or surgical biopsy. In particular embodiments, the surgical biopsy is an incisional biopsy, which removes only part of the suspicious area. In other embodiments, the surgical biopsy is an excisional biopsy, which removes the entire diseased tissue (e.g., tumor) or abnormal area. In particular embodiments, an excisional tumor tissue sample is obtained from a tumor that has been excised with the intent to “cure” a patient in the case of early stage disease, wherein in other embodiments, the excisional tumor tissue sample is obtained from an excised bulk of primary tumor in later stage disease. Tumor tissue samples may include primary tumor tissue, metastatic tumor tissue and/or secondary tumor tissue. Tumor tissue samples may be cell cultures, e.g., cultures of tumor-derived cell lines. In certain embodiments, a tissue section is a cell line, e.g., a cell pellet of a cultured cell line, such as a tumor cell line. In particular embodiments, the cell line or cell pellet is frozen or was previously frozen. Such cell lines and pellets are useful, e.g., as positive or negative controls for imaging with various reagents. Tumor tissue samples may also be xenograft tumors, e.g., tumors obtained from animals administered with tumor cells, e.g., a human tumor cell line. In certain embodiments, a first tumor tissue sample from a subject is a primary tumor tissue sample obtained during an initial surgery intended to remove the entire tumor, and a second tumor tissue sample is obtained from the same subject is a metastatic tumor tissue sample or a secondary tumor tissue sample obtained during a later surgery.

Tissue sections, e.g., tumor tissue samples, may be obtained surgically or using a laparoscope. A tissue section may be a tissue sample obtained from any part of the body to examine it for disease or injury, e.g., presence of cancer tissue or cells, or the extent or characteristics thereof. In particular embodiments, the tissue section includes abdominal tissue, bone, bone marrow, breast tissue, endometrial tissue, kidney tissue, liver tissue, lung or chest tissue, lymph node, nerve tissue, skin, testicular tissue, head or neck tissue, or thyroid tissue. In certain embodiments, the tissue is obtained from brain, breast, skin, bone, joint, skeletal muscle, smooth muscle, red bone marrow, thymus, lymphatic vessel, thoracic duct, spleen, lymph node, nasal cavity, pharynx, larynx, trachea, bronchus, lung, oral cavity, esophagus, liver, stomach, small intestine, large intestine, rectum, anus, spinal cord, nerve, pineal gland, pituitary gland, thyroid gland, thymus, adrenal gland, pancreas, ovary, testis, heart, blood vessel, kidney, uterus, urinary bladder, urethra, prostate gland, penis, prostate, testis, scrotum, ductus deferens, mammary glands, ovary, uterus, vagina, or uterine tube.

In embodiments, a tissue section has a size greater than sections typically examined by traditional pathology thin section or immunohistochemical analysis, which are typically in the range of 4-10 microns thick. In certain embodiments, a tissue section is greater than 20 microns, greater than 50 microns, greater than 100 microns, greater than 200 microns, greater than 500 microns, greater than 1 mm, greater than 2 mm, greater than 5 mm, greater than 10 mm or greater than 20 mm in thickness and/or length. In particular embodiments, the tissue section has a length and/or a thickness between 20 microns and 20 mm, between 20 microns and 10 mm, or between 50 microns and 1 mm. In certain embodiments, a tissue section is a cubic sample with each side greater than 10 microns, greater than 20 microns, greater than 50 microns, greater than 100 microns, greater than 200 microns, greater than 500 microns, greater than 1 mm, greater than 2 mm, greater than 5 mm, greater than 10 mm, or greater than 2 mm in thickness and/or length. In some embodiments, a tissue section is thinner, e.g., from about 4-10 or 4-20 microns in thickness.

In embodiments, the method further includes removing the embedding material. In embodiments, the method further includes removing the embedding material. For example, if the embedding material is paraffin wax, the embedding material is removed by contacting the sample-carrier construct with a hydrocarbon solvent, such as xylene or hexane, followed by two or more washes with decreasing concentrations of an alcohol, such as ethanol.

In embodiments, the solid support includes an IR reflective coating. In embodiments, the IR reflective coating is attached to the solid support. In embodiments, the IR reflective coating is attached to the solid support, wherein the IR reflective coating is in contact with the polymer described herein. In embodiments, the IR reflective coating includes metal oxides. In embodiments, the IR reflective coating includes titanium dioxide, zinc oxide, tin oxide, tantalum pentoxide, silicon dioxide, indium tin oxide, silver-based coating, ceramic-based coating or a combination thereof. In embodiments, the IR reflective coating includes SiO₂, TiO₂, Al₂O₃ and Ta₂O₅ and fluorides such as MgF₂, LaF₃ and AlF₃. In embodiments, the IR reflective coating includes tantalum pentoxide (Ta₂O₅) and silicon dioxide (SiO₂). In embodiments, the infrared (IR) reflective coating includes one or more layers of silicon dioxide (SiO₂) and tantalum pentoxide (Ta₂O₅). In embodiments, the infrared (IR) reflective coating includes alternating layers of silicon dioxide (SiO₂) and tantalum pentoxide (Ta₂O₅), wherein the layer of silicon dioxide (SiO₂) is in direct or indirect contact with the polymer (e.g., the polymer described herein). In embodiments, the infrared (IR) reflective coating includes alternating layers of silicon dioxide (SiO₂) and tantalum pentoxide (Ta₂O₅), wherein the layer of tantalum pentoxide (Ta₂O₅) is in direct or indirect contact with the polymer (e.g., the polymer described herein).

In embodiments, the IR reflective coating reflects near-infrared radiation (NIR). In embodiments, the IR reflective coating reflects mid- or far-infrared radiation. In embodiments, the IR reflective coating reflects wavelengths greater than 750 nm. In embodiments, the IR reflective coating reflects wavelengths greater than 760 nm. In embodiments, the IR reflective coating reflects wavelengths greater than 770 nm. In embodiments, the IR reflective coating reflects wavelengths greater than 780 nm. In embodiments, the IR reflective coating reflects wavelengths greater than 790 nm. In embodiments, the IR reflective coating reflects wavelengths greater than 800 nm. In embodiments, the IR reflective coating reflects wavelengths from about 750 nm to 1,000 μm. In embodiments, the infrared (IR) reflective coating includes one or more layers of silicon dioxide (SiO₂) and tantalum pentoxide (Ta₂O₅). A multilayer configuration leverages the distinct optical properties of both materials to enhance the IR reflectivity. Silicon dioxide, known for its low refractive index, and tantalum pentoxide, recognized for its high refractive index, are alternately layered to create a stack that exhibits high reflectance in the infrared spectrum. The alternating layers of SiO₂ and Ta₂O₅ result in constructive interference of light at specific wavelengths, thereby enhancing the IR reflective capability of the coating. The number and thickness of these layers can be tailored to target specific wavelengths within the IR range, or permitting a certain percentage of radiation to transmit. For example, the IR reflective coating may reflect 2-3%, 2-6%, or 2 to 10% of the total IR radiation, and it absorbs or transmits the remaining IR radiation (e.g., greater than about 90% of the IR radiation). In embodiments, the IR reflective coating reflects about 1%, about 2%, about 3%, about 4%, about 5%, about 6%, about 7%, about 8%, about 9%, or about 10% of the total IR radiation.

In embodiments, the IR reflective coating aids autofocus mechanisms in optical instruments (e.g., fluorescence microscopy instruments) to provide consistent signal across various z-heights (e.g., the depth of an image). In embodiments, the IR reflective coating increases the amount of light reflected to the autofocus sensor to provide consistent signal across various z-heights. In embodiments, the IR reflective coating improves the signal to noise ratio of an image acquired by an optical instrument.

In embodiments, the solid support includes polymerized units of alkoxysilyl methacrylate, alkoxysilyl acrylate, alkoxysilyl methylacrylamide, alkoxysilyl acrylamide, or a copolymer thereof. In embodiments, the resist is a polydimethylsiloxane (PDMS), poly(methyl methacrylate) (PMMA), cyclic olefin copolymer (COC), silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, SU-8 resist, or an organically modified ceramic polymer resist.

In embodiments, the solid support includes a photoresist. In embodiments, the solid support includes an organically modified ceramic polymer resist. In embodiments, the resist is an organically modified ceramic polymer resist. In embodiments, the organically modified ceramic polymer resist includes polymerized alkoxysilyl methacrylate polymers and metal oxides. In embodiments, the organically modified ceramic polymer resist includes polymerized alkoxysilyl acrylate polymers and metal oxides. In embodiments, the solid support includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

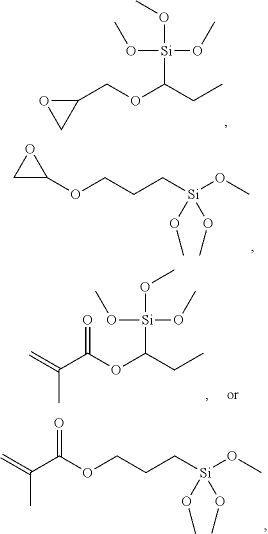

or a copolymer thereof.

The photoresist (alternatively referred to as a resist) is an active material layer that can be patterned by selective exposure and must “resist” chemical/physical attach of the underlying substrate. A photoresist is a light-sensitive polymer material used to form a patterned coating on a surface. The process begins by coating a substrate (e.g., a glass substrate) with a light-sensitive organic material. A mask with the desired pattern is used to block light so that only unmasked regions of the material will be exposed to light. In the case of a positive photoresist, the photo-sensitive material is degraded by light and a suitable solvent will dissolve away the regions that were exposed to light, leaving behind a coating where the mask was placed. In the case of a negative photoresist, the photosensitive material is strengthened (either polymerized or cross-linked) by light, and a suitable solvent will dissolve away only the regions that were not exposed to light, leaving behind a coating in areas where the mask was not placed. In embodiments, the solid support includes an epoxy-based photoresist (e.g., SU-8, SU-8 2000, SU-8 3000, SU-8 GLM2060). In embodiments, the solid support includes a negative photoresist. Negative refers to a photoresist whereby the parts exposed to UV become cross-linked (i.e., immobilized), while the remainder of the polymer remains soluble and can be washed away during development.

In embodiments, the solid support includes a glass substrate having a surface coated in silsesquioxane resist (e.g., polyhedral oligosilsesquioxanemethacrylate (POSS)), an epoxy-based polymer resist (e.g., SU-8 as described in U.S. Pat. No. 4,882,245), poly(vinylpyrrolidone-vinyl acrylic acid) copolymer resist (e.g., as described in U.S. Pat. No. 7,467,632), or novolaks resist, bisazides resist, or a combination thereof (e.g., as described in U.S. Pat. No. 4,970,276). In embodiments, the resist is removed prior to loading.

A “resist” as used herein is used in accordance with its ordinary meaning in the art of lilthography and refers to a polymer matrix (e.g., a polymer network). In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the solid support includes a Hydrogen Silsesquioxane (HSQ) polymer (e.g., HSQ resist). In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, A1, Ti or Ta atoms.

In embodiments, the solid support includes a resist (e.g., a nanoimprint lithography (NIL) resist). Nanoimprint resists can include thermal curable materials (e.g., thermoplastic polymers), and/or UV-curable polymers. In embodiments, the solid support is generated by pressing a transparent mold possessing the pattern of interest (e.g., the pattern of wells) into photo-curable liquid film, followed by solidifying the liquid materials via a UV light irradiation. Typical UV-curable resists have low viscosity, low surface tension, and suitable adhesion to the glass substrate. For example, the solid support surface is coated in an organically modified ceramic polymer (ORMOCER®, registered trademark of Fraunhofer-Gesellschaft zur Forderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, US 2010/0160478, or U.S. Pat. No. 10,268,096 B2, each of which is incorporated herein by reference. In embodiments, the solid support surface is coated in an organically modified ceramic polymer including (ORMOCER®, registered trademark of Fraunhofer-Gesellschaft zur Forderung der angewandten Forschung e. V. in Germany). In embodiments, the solid support surface is coated in an organically modified ceramic polymer wherein the organically modified ceramic polymer includes an inorganic-organic hybrid polymer that includes Si—O bonds. In embodiments, the solid support surface is coated in an organically modified ceramic polymer wherein the organically modified ceramic polymer includes an inorganic-organic hybrid polymer that includes Si—C bonds. In embodiments, the solid support surface is coated in an organically modified ceramic polymer wherein the organically modified ceramic polymer includes free acrylate moieties. In embodiments, the polymer is an organically modified ceramic polymer wherein the organically modified ceramic polymer includes an inorganic-organic hybrid polymer that includes Si—O bonds. In embodiments, polymer is an organically modified ceramic polymer wherein the organically modified ceramic polymer includes an inorganic-organic hybrid polymer that includes Si—C bonds. In embodiments, the polymer is an organically modified ceramic polymer wherein the organically modified ceramic polymer includes free acrylate moieties. In embodiments, the polymer contains organically crosslinked heteropolysiloxane moieties.

In some embodiments, the solid support includes a hydrophobic polymer layer. In embodiments, the solid support includes a perfluorinated polymer. In embodiments, the solid support includes a polyfluorinated polymer. In embodiments, the solid support includes polymerized units of a fluorine-containing methacrylate (e.g., CH₂═C(CH₃)COOC—(CF₃)₂CF₂CF₂CF₃). Non-limiting examples and synthetic protocols of fluorine-containing methacrylate monomers may be found in Zhang, D., (2018). Materials (Basel, Switzerland), 11(11), 2258 (2018), which is incorporated herein by reference. In embodiments, the fluorinated polymer is an amorphous (non-crystalline) fluoropolymer (e.g., CYTOP® from Bellex), a crystalline fluoropolymer, or a fluoropolymer having both amorphous and crystalline domains.

In embodiments, the first solid support includes a refractive index of about 1.5. In embodiments, the first solid support includes a refractive index of about 1.52. In embodiments, the first solid support includes a refractive index of about 1.524 to about 1.527. In embodiments, the second solid support includes a refractive index of about 1.5. In embodiments, the second solid support includes a refractive index of about 1.52. In embodiments, the second solid support includes a refractive index of about 1.524 to about 1.527.

In embodiments, the first solid support includes drilled ports. In embodiments, the second solid support includes drilled ports. In embodiments, the second solid support includes a channel bored into the second solid support. In embodiments, the second solid support includes a gasket, wherein the gasket defines the reaction chamber. In embodiments, the second solid support includes 2, 3, 4, 5, 6, 7, 8, 9, 10, 11, or 12 reaction chambers. In embodiments, the first solid support includes an inlet port and an outlet port. In embodiments, the second solid support includes an inlet port and an outlet port.

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissues include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv©, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

The biological sample can be a biological tissue, cultured cells, or cells taken from an animal subject of interest. In embodiments, the biological sample includes material that is human origin or mouse origin. In embodiments, the biological sample is fresh, frozen, or fixed. In embodiments it can be a section or core obtained from a formalin-fixed paraffin-embedded (FFPE) tissue block. The sample can include material from a tissue section, tissue micro-array (TMA), cell pellet, core biopsy, needle biopsy, or cells obtained from a blood or serum sample. In embodiments, the biological sample is immobilized on a surface of a functionalized slide, a functionalized plate, a functionalized well, or a functionalized film. The biological sample can be contacted with two or more (e.g., 3 or more, 4 or more, 5 or more, 6 or more, 8 or more, 10 or more, 12 or more, 15 or more, 20 or more, 30 or more, 40 or more, 50 or more, 60 or more, 80 or more, or even more) antibodies, antibody fragments, or a combination thereof. The sample can be contacted with a cocktail of all the antibodies or antibody fragments, or combinations of multiple subsets of the total number of antibodies.

In an aspect is provided a system for detecting biomolecules in a cell or tissue, the system including: a substrate including a channel (e.g., a flow cell), wherein the cell or tissue is immobilized within the channel; a first oligonucleotide including a cleavable site, wherein cleaving the first oligonucleotide at the cleavable site produces a first fragment and a second fragment, each having a melting temperature lower than a melting temperature of the intact first oligonucleotide; a second oligonucleotide; a cleaving agent configured to cleave the cleavable site of the first oligonucleotide; a polymerase; and a plurality of labeled nucleotides, wherein the system is configured to sequentially: (i) hybridize the first oligonucleotide to a first target polynucleotide in the cell or tissue and detect a signal associated with the first oligonucleotide, (ii) cleave the first oligonucleotide at the cleavable site and remove the first fragment and the second fragment, and (iii) hybridize the second oligonucleotide to a second target polynucleotide in the cell or tissue and detect a signal associated with the second oligonucleotide. In embodiments, the system includes a fluidic system in fluid communication with the channel, wherein the fluidic system is configured to deliver the first oligonucleotide, the second oligonucleotide, the cleaving agent, the polymerase, and the plurality of labeled nucleotides to the channel. In embodiments, the system includes an imaging system configured to detect the signal associated with the first oligonucleotide and the signal associated with the second oligonucleotide. In embodiments, the imaging system includes a fluorescence excitation source.

In embodiments, the detection apparatus (alternatively referred to as an imaging system) includes at least one camera. In embodiments, the detection apparatus includes at least two cameras. For example, the detection apparatus includes a first camera and a second camera and wherein the first camera and the second camera are each configured to obtain an image of two different color channels. In embodiments, the first camera collects an image of first and second fluorescent channels and the second camera collects an image of third and fourth fluorescent channels. In embodiments, a fluorescent channel refers to a specified range of wavelengths within the electromagnetic spectrum, typically corresponding to a distinct color of light. Each channel is used to capture or detect signals from a particular fluorescent dye or fluorophore that emits light at a unique wavelength when excited by a light source. In multi-channel fluorescence imaging, different fluorophores are used to label various targets within a sample, with each fluorophore emitting light within a defined wavelength range (channel). The channels are spectrally distinct, meaning that they do not overlap significantly in the wavelengths they cover, allowing for the independent detection of multiple fluorophores in a single sample.

In embodiments, imaging includes repeated detecting emissions in different z planes, for example using a step size in the z-axis. In embodiments, the step size in the z-axis is about 50 nm, about 100 nm, about 200 nm, about 300 nm, about 400 nm, about 500 nm, about 600 nm, about 700 nm, about 800 nm, about 900 nm, or about 1000 nm. In embodiments, the step size in the Z-axis is about 50 nm, about 100 nm, about 200 nm, about 300 nm, or about 400 nm.

In embodiments, the method includes obtaining images of the tissue section along a detection axis. In embodiments, the method includes obtaining images of the tissue section along a z-axis, wherein the tissue section is on an image plane (e.g., an xy plane). In embodiments, the method includes obtaining images of the tissue section along the z-axis with a step size of about 50 nm, about 100 nm, about 200 nm, about 300 nm, about 400 nm, about 500 nm, about 600 nm, about 700 nm, about 800 nm, about 900 nm, or about 1000 nm. In embodiments, the method includes obtaining images of the tissue section along the z-axis with a step size of about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm. In embodiments, the method includes obtaining images of the tissue section along the z-axis with a step size of about 50 nm. In embodiments, the method includes obtaining images of the tissue section along the z-axis with a step size of about 100 nm. In embodiments, the method includes obtaining images of the tissue section along the z-axis with a step size of about 150 nm. In embodiments, the method includes obtaining images of the tissue section along the z-axis with a step size of about 200 nm. In embodiments, the method includes obtaining images of the tissue section along the z-axis with a step size of about 250 nm. In embodiments, the method includes obtaining images of the tissue section along the z-axis with a step size of about 300 nm. In embodiments, the method includes obtaining images of the tissue section along the z-axis with a step size of about 350 nm. In embodiments, the method includes obtaining images of the tissue section along the z-axis with a step size of about 400 nm.

In embodiments, the system includes one or more image sensors (or cameras) that may be the same or may be different, and may include any of a variety of image sensors including but not limited to, photodiode arrays, charge-coupled device (CCD) sensors or cameras, or complementary metal-oxide-semiconductor (CMOS) image sensors or cameras. In embodiments, the one or more image sensors may comprise one-dimensional (linear) or two-dimensional pixel array sensors. In embodiments, the one or more image sensors may comprise monochrome image sensors (e.g., configured to capture greyscale images) or color image sensors (e.g., configured to capture RGB or color images). In embodiments, the one or more image sensors may be used to capture single images, e.g., a single image for each cycle of a plurality of cycles. In embodiments, the one or more image sensors may be used to capture a series of images, e.g., a series of images during each cycle of a plurality of cycles. In embodiments, a series of images may include images (or video frames) that correspond to images captured before, during, and/or after an event, e.g., before, during, and/or after addition of a probe to the sample being imaged. In embodiments, a series of images includes 2 images, 3 images, 4 images, 5 images, 10 images, 20 images, 30 images, 40 images, 50 images, 100 images, 200 images, 300 images, 400 images, 500 images, 1,000 images, or more than 1,000 images.

In embodiments, the system is configured to perform volumetric imaging (or optical sectioning). In embodiments, the imaging includes the acquisition of a plurality (or “stack”) of two-dimensional (2D) images to form a three-dimensional (3D) representation of the sample, where each two-dimensional image is aligned with the other images of the plurality in the sample plane (e.g., the X-Y plane), but is offset from the other two-dimensional images in a direction parallel to the optical axis of the imaging module (e.g., in the Z-direction). In embodiments, the stack of images may be acquired sequentially. In embodiments, the stack of images may be acquired simultaneously.

In embodiments, the system is configured to control the delivery of fluids such as reagents and/or buffers to a sample, e.g., a sample contained within the flow cell. In embodiments, the one or more fluidics controllers may be configured to control volumetric flow rates for one or more fluids or reagents, linear flow velocities for one or more fluids or reagents, mixing ratios for one or more fluids or reagents, or any combination thereof. Fluidics modules may include one or more fluid flow sensors (e.g., flow rate sensors, pressure sensors, etc.), one or more fluid flow actuators (e.g., pumps), one or more fluid flow control devices (e.g., valves), one or more processors (and associated electronics), tubing and connectors to connect the one or more fluidics modules to one or more flow cells, or any combination thereof

In embodiments, the system includes one or more processing units CPU(s) (also referred to as processors), one or more network interfaces, a user interface including a display and an input module, a non-persistent, a persistent memory, and one or more communication buses for interconnecting these components. The one or more communication buses optionally include circuitry (sometimes called a chipset) that interconnects and controls communications between system components. The non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory optionally includes one or more storage devices remotely located from the CPU(s). The persistent memory, and the non-volatile memory device(s) within the non-persistent memory, comprise non-transitory computer readable storage medium. In embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In embodiments, the computing device includes memory in electronic communication with the processor. The memory architecture may include at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, and data structures that perform particular tasks. A computing device can optionally communicate with one or more external devices such as a keyboard, a pointing device (e.g., a mouse), a display, such as a graphical user interface (GUI), or other device that facilitates interaction of a use with the unmanned autonomous vehicle. Similarly, the computing device can communicate with other devices (e.g., via network card, modem, etc.). Such communication can occur via I/O interfaces. In embodiments, the computing system may communicate with one or more networks such as a local area network (LAN), a general wide area network (WAN), and/or a public network (e.g., the Internet) via a suitable network adapter.

In embodiments, the systems and devices generate data that may be used to form an image. In embodiments, the image includes a 2D or 3D representation of the tissue. In some embodiments, one or more of the images include an image of other analytes, such as proteins in a biological sample. In some embodiments, an image is acquired using transmission light microscopy (e.g., bright field transmission light microscopy, dark field transmission light microscopy, oblique illumination transmission light microscopy, dispersion staining transmission light microscopy, phase contrast transmission light microscopy, differential interference contrast transmission light microscopy, emission imaging, etc.). In embodiments, the image is in any file format including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CDS, CPT, PSD, PSP, XCF, PDN, CGM, SVG, PostScript, PCT, WMF, EMF, SWF, XAML, and/or RAW. In embodiments, the image is represented as an array (e.g., matrix) comprising a plurality of pixels, such that the location of each respective pixel in the plurality of pixels in the array (e.g., matrix) corresponds to its original location in the image. In some embodiments, an image is represented as a vector comprising a plurality of pixels, such that each respective pixel in the plurality of pixels in the vector comprises spatial information corresponding to its original location in the image.

In embodiments, a pixel includes one or more pixel values (e.g., intensity value). In embodiments, each respective pixel in the plurality of pixels includes one pixel intensity value, such that the plurality of pixels represents a single-channel image comprising a one-dimensional integer vector comprising the respective pixel values for each respective pixel. For example, an 8-bit single-channel image (e.g., grey-scale) can include 2⁸ or 256 different pixel values (e.g., 0-255). In embodiments, each respective pixel in the plurality of pixels of an image includes a plurality of pixel values, such that the plurality of pixels represents a multi-channel image comprising a multi-dimensional integer vector, where each vector element represents a plurality of pixel values for each respective pixel. For example, a 24-bit 3-channel image (e.g., RGB color) can include 2²⁴ (e.g., 2^{8×3}) different pixel values, where each vector element comprises 3 components, each between 0-255. In some embodiments, an n-bit image includes up to 2ⁿ different pixel values, where n is any positive integer.

In embodiments, each pixel in the plurality of pixels of the image has a pixel size (resolution) between 0.8 pm and 4.0 pm. In embodiments the pixel size is derived by dividing the camera pixel size (resolution) by the magnification of the objective lens of the camera used to capture values for the plurality of pixels. In embodiments, each pixel in the plurality of pixels has a pixel size between 0.4 pm and 5.0 pm. In embodiments, each pixel in the plurality of pixels of the image has a pixel size (resolution) between 0.8 pm and 4.0 pm or between 0.4 pm and 5.0 pm.

In embodiments, the data processor provides the image for display via a display of the computing device. In embodiments, the image is provided for display via a GUI configured within the display of the computing device. In embodiments, the data processor receives an input identifying one or more modifications and/or one or more image analysis steps based on the provided image. For example, the display of the computing device can include a touchscreen display configured to receive a user input identifying a respective pattern of an image of the biological sample on the displayed image. In embodiments, the GUI can be configured to receive a user provided input identifying the modifications and/or one or more image analysis steps.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre compiled or as-compiled fashion.

Examples of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media (e.g., computer-readable media) include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for tissue sample analysis. Examples of Us include, without limitation, a graphical user interface (GUI) and web-based user interface. Methods and systems of the present disclosure can be implemented by way of one or more algorithms. An algorithm can be implemented by way of software upon execution by the central processing unit.

According to certain embodiments, the above-described data feeds may be stored in databases such as database servers that store master data as well as logging and trace information. The databases may also provide an API and/or API access (e.g., for open source) to the web server for data interchange based on JSON specifications. According to certain embodiments, the database servers may be optimally designed for storing large amounts of data, responding quickly to incoming requests, having a high availability and historizing master data.

The system contemplates uses in association with web services, utility computing, pervasive and individualized computing, security and identity solutions, autonomic computing, cloud computing, commodity computing, mobility and wireless solutions, open source, biometrics, grid computing, and/or mesh computing.

In an aspect is provided a flow cell assembly. In embodiments, the flow cell assembly includes a first solid support; a polymer attached to the first solid support; a cell or tissue attached to the polymer; a second solid support attached to the first solid support, wherein the second solid support is configured to define a reaction chamber. In embodiments, the second solid support is configured to define a reaction chamber when attached to the first solid support. In embodiments, the flow cell assembly includes a frame configured to retain the flow cell assembly. Suitable flow cell frames and handles are described in U.S. Pat. No. 11,747,262. The frame can be configured to retain the flow cell such that a maximal surface area of the flow cell can be available to be exposed to an optical lens (e.g., the optical lens of a nucleic acid sequencing device). The optical lens (e.g., the optical lens of the sequencing device) can be configured to detect excitation, emission, or other signals present on the flow cell. The frame can be configured to retain the flow cell such that a maximal surface area of the flow cell can be available to be in contact with the receiver of a nucleic acid sequencer. The retaining of the flow cell further can include constraining a first, a second, a third, a fourth, a fifth, and a sixth degree of freedom of the flow cell. The frame can be an injection molded frame. The handle can be a raised handle. The frame can be further configured to provide a gap between a work surface and the flow cell. The frame further can include at least one ferromagnetic pin. The at least one biasing feature can be a spring finger. The at least one biasing feature can be a tab. The flow cell can further include a microchip. The microchip can be an electronically erasable programmable read only memory (EEPROM) chip. Flow cells provide a convenient format for housing an array of clusters produced by the methods described herein, in particular when subjected to an SBS or other detection technique that involves repeated delivery of reagents in cycles.

In embodiments, the first solid support includes one or more channel(s). In embodiments, the first solid support includes a channel bored into the first solid support. In embodiments, the first solid support includes a plurality of channels bored into the first solid support. In embodiments, the first solid support includes 2 channels bored into the first solid support. In embodiments, the first solid support includes 3 channels bored into the first solid support. In embodiments, the first solid support includes 4 channels bored into the first solid support. In embodiments, the width of the channel is from about 1 to 5 mm. In embodiments, the width of the channel is from about 5 to 10 mm. In embodiments, the width of the channel is from about 10 to 15 mm. In embodiments, the width of the channel is from about 5 mm. In embodiments, the width of the channel is from about 11 mm. In embodiments, the second solid support includes one or more channel(s). In embodiments, the second solid support includes a channel bored into the second solid support. In embodiments, the second solid support includes a plurality of channels bored into the second solid support. In embodiments, the second solid support includes 2 channels bored into the second solid support. In embodiments, the second solid support includes 3 channels bored into the second solid support. In embodiments, the second solid support includes 4 channels bored into the second solid support. In embodiments, the width of the channel is from about 1 to 5 mm. In embodiments, the width of the channel is from about 5 to 10 mm. In embodiments, the width of the channel is from about 10 to 15 mm. In embodiments, the width of the channel is from about 5 mm. In embodiments, the width of the channel is from about 11 mm.

In embodiments, the second solid support includes a gasket (alternatively referred to herein as a spacer), wherein the gasket defines the reaction chamber. In embodiments, the gasket defines a perimeter of a channel. In embodiments, the gasket includes silicone, polyimide, fluorocarbon elastomer, ethylene propylene diene, polychloroprene, polytetrafluoroethylene, nitrile rubber, butyl rubber, natural rubber, thermoplastic elastomer, or a combination thereof. In embodiments, the second solid support includes a spacer element to form an offset surface. In embodiments, the second solid support includes one or more channels. The channel(s) may be formed by affixing a spacer element to create a defined gap or channel through which liquid can flow or be contained. The spacer element may be made of any suitable material, for example resin, glass, plastic, silicon, an adhesive, or a combination thereof. In embodiments, the spacer element includes a first adhesive in contact with the functionalized glass slide and second adhesive in contact with the second solid support. In embodiments, the spacer element includes a first adhesive in contact with the functionalized glass slide, a second adhesive in contact with the second solid support, and a carrier material in contact with the first adhesive and the second adhesive. The depth of the resulting channel may be controlled by including a carrier material (e.g., one or more polymer or copolymer layers) between the adhesives. In embodiments, the spacer element may form the walls of the reaction chamber, wherein the reaction chamber includes the sample. In embodiments, the spacer element is further attached to the copolymer attached the polymer of the first solid support. In embodiments, the gasket is referred to as a spacer element.

In embodiments, the flow cell assembly further includes 2, 3, 4, 5, 6, 7, 8, 9, 10, 11, or 12 reaction chambers (e.g., channels). In embodiments, the flow cell assembly includes 2 distinct reaction chambers (e.g., channels). In embodiments, the flow cell assembly includes 4 distinct reaction chambers (e.g., channels). In embodiments, each reaction chamber includes a depth of about 50 μm to about 150 μm. In embodiments, the reaction chamber includes a depth of about 80 μm to about 110 μm. In embodiments, the reaction chamber includes a width of about 4 μm to about 15 μm.

In another aspect, provided herein is a kit including reagents for performing multiplexed in situ analysis of biomolecules in a cell or tissue. In embodiments, the kit includes a first sequencing primer and a second sequencing primer, wherein at least one of the sequencing primers includes a cleavable site. In embodiments, the kit further includes a polymerase capable of incorporating labeled nucleotides, a ligase for padlock probe circularization, and a set of padlock probes designed to hybridize to distinct target nucleic acid sequences. In embodiments, the kit includes a cleavage reagent for selectively removing cleaved primer fragments.

In embodiments, the kit includes the composition as described herein. In embodiments, the kit includes labeled nucleotides including differently labeled nucleotides, enzymes, buffers, oligonucleotides, and related solvents and solutions. In embodiments, the kit includes a padlock probe (e.g., a polynucleotide as described herein or polynucleotide probe described herein). In embodiments, the kit includes a specific binding agent described herein, wherein the specific binding agent includes an oligonucleotide described herein. The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using chemical ligation techniques. In embodiments, the kit includes components useful for circularizing template polynucleotides using a ligation enzyme (e.g., CircLigase™ enzyme, Taq DNA Ligase, HiFi Taq DNA Ligase, T4 ligase, or Ampligase® DNA Ligase). In embodiments the ligation enzyme is an RNA-dependent DNA ligase (e.g., SplintR® ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 ligase, or Ampligase™ DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof, a thymine nucleotide; a cytosine nucleotide, or analog thereof, and a guanine nucleotide, or analog thereof may be labeled with a different fluorescent label.

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

In an aspect is provided a kit including a circularizable probe (e.g., a polynucleotide probe described herein), a ligase, and a specific binding agent attached to a target polynucleotide, wherein the circularizable probe includes a first hybridization sequence capable of hybridizing to a first sequence of a target polynucleotide, a second hybridization sequence capable of hybridizing to a second sequence of the target polynucleotide. In embodiments, the circularizable probe includes a barcode sequence. In embodiments, the target polynucleotide is an oligonucleotide described herein.

### III. Methods

In an aspect is provided a method of detecting biomolecules (e.g., proteins, organelles, and/or nucleic acids) in a cell or tissue. The method includes hybridizing a first oligonucleotide to a first target polynucleotide in the cell or tissue, wherein the first oligonucleotide includes a cleavable site. The method further includes detecting a first detectable label associated with the first oligonucleotide. In embodiments, the first detectable label is a fluorescent label. In embodiments, the first detectable label is covalently attached to the first oligonucleotide. In embodiments, the first detectable label is associated with a nucleotide that is incorporated into or bound to the first oligonucleotide. Following detection of the first detectable label, the method includes cleaving the first oligonucleotide at the cleavable site to produce a first fragment and a second fragment. In embodiments, each fragment has a melting temperature lower than a melting temperature of the first oligonucleotide. The reduction in melting temperature of the fragments relative to the intact first oligonucleotide facilitates dissociation of the fragments from the first target polynucleotide under conditions that would not dissociate the intact first oligonucleotide. In embodiments, the melting temperature of each fragment is at least 5° C., at least 10° C., at least 15° C., or at least 20° C. lower than the melting temperature of the intact first oligonucleotide. In embodiments, the cleavable site is positioned within the first oligonucleotide such that upon cleavage, the resulting first fragment and second fragment each have a melting temperature below the temperature at which subsequent method steps are performed.

In embodiments, the method further includes removing the first fragment and the second fragment. In embodiments, removing the first fragment and the second fragment includes washing the cell or tissue under conditions sufficient to dissociate the fragments from the first target polynucleotide. In embodiments, removing includes incubating the cell or tissue at a temperature above the melting temperature of the first fragment and the second fragment. In embodiments, removing includes contacting the cell or tissue with a wash buffer at a temperature of about 55° C. to about 65° C. In embodiments, removing includes contacting the cell or tissue with a wash buffer at a temperature of about 60° C. Following removal of the first fragment and the second fragment, the method includes hybridizing a second oligonucleotide to a second target polynucleotide in the cell or tissue. The method further includes detecting a second detectable label associated with the second oligonucleotide. In embodiments, the second detectable label is a fluorescent label. In embodiments, the second oligonucleotide includes a cleavable site. In embodiments, the second oligonucleotide does not include a cleavable site.

In embodiments, removing includes a fluid exchange. For example, removing may include washing the cell or tissue with a wash buffer, such as a saline-sodium citrate (SSC) buffer or a phosphate buffered saline (PBS) buffer, to facilitate the removal of cleaved fragments from the cell or tissue. In some embodiments, removing may include flowing a denaturing solution over the cell or tissue to promote dissociation of the cleaved fragments from the target polynucleotide. Suitable denaturing solutions may include a dilute sodium hydroxide solution (e.g., about 0.02M NaOH), formamide (e.g., about 50% to about 100% formamide), or a combination thereof. In some embodiments, removing may include flowing a solution comprising formamide at a concentration of about 10%, about 25%, about 50%, about 75%, or about 100% over the cell or tissue. In some embodiments, removing may include a series of fluid exchanges, for example, a first fluid exchange comprising a denaturing solution followed by a second fluid exchange comprising a wash buffer. In some embodiments, removing may include flowing a heated wash buffer over the cell or tissue, wherein the temperature of the wash buffer is at or above the melting temperature of the cleaved fragments. In some embodiments, the fluid exchange is performed within a channel of a flow cell or a microfluidic device, wherein the denaturing solution or wash buffer is delivered through the channel by a fluidic delivery system. In some embodiments, removing does not require a change in temperature, as the cleaved fragments have a melting temperature below the reaction temperature and spontaneously de-hybridize from the target polynucleotide upon cleavage.

In an aspect is provided a method of detecting polynucleotides, the method including: (i) hybridizing a first sequencing primer to a first polynucleotide, incorporating with a polymerase a first labeled nucleotide into the first sequencing primer, and detecting the first labeled nucleotide; (ii) cleaving the first sequencing primer into a first fragment and a second fragment and removing the fragments; and (iii) hybridizing a second sequencing primer to a second polynucleotide, incorporating with a polymerase a second labeled nucleotide into the second sequencing primer, and detecting the second labeled nucleotide, wherein the first polynucleotide and the second polynucleotide are in a cell or tissue.

In embodiments, the polynucleotide is associated with a biomolecule. For example, as described herein, the polynucleotide is generated via an amplification reaction of a probe-specific oligonucleotide probe (e.g., a padlock probe). In embodiments, the biomolecule is a lipid, carbohydrate, peptide, protein, or antigen binding fragment. In embodiments, the biomolecule is a lipid. In embodiments, the biomolecule is a carbohydrate. In embodiments, the biomolecule is a peptide. In embodiments, the biomolecule is a protein. In embodiments, the biomolecule is an antigen binding fragment. In embodiments, the biomolecule is an oligonucleotide. In embodiments, the target is a nucleic acid. In embodiments, the target is a non-nucleic acid target. Non-nucleic acid targets include, but are not limited to, lipids, carbohydrates, peptides, proteins, glycoproteins, lipoproteins, phosphoproteins, acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In embodiments, the target is inside a cell or on a cell surface, such as a transmembrane analyte or one that is attached to the cell membrane. In embodiments, the target is an organelle (e.g., nuclei or mitochondria).

In embodiments, the method includes immobilizing a cell or tissue onto a substrate described herein, wherein the cell or tissue includes the target molecule or target biomolecule to be detected. In embodiments, the method includes immobilizing a plurality of tissues onto a substrate described herein, wherein the tissue includes the target molecule or target biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 mm×4 mm sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto a substrate or support as described herein (e.g., inverting the punch device); pushing the sample portion out of the punch device using a piston, so that all or a portion thereof of the sample portion is positioned on a substrate or support as described herein. In embodiments, the method further includes cutting a sample portion from the biological sample using two or more punch devices such that each punch device contains a different the sample portion; mounting each punch device containing the sample portion onto a substrate or support as described herein; pushing the sample portions out of the punch devices using one or more pistons so that the sample portions are positioned onto a substrate or support as described herein.

In embodiments, (i), (ii), and (iii) occur at about 55° C. to about 65° C. In embodiments, (i), (ii), and (iii) occur at about 58° C. to about 62° C. In embodiments, (i), (ii), and (iii) occur at about 60° C. In embodiments, (i), (ii), and (iii) occur at substantially the same temperature. In embodiments, the method is performed under isothermal conditions, wherein steps (a), (b), and (c) occur at substantially the same temperature. This isothermal workflow eliminates the need for temperature cycling between hybridization, cleavage, and subsequent detection steps. By maintaining a consistent temperature throughout the process, the method simplifies instrumentation requirements and reduces the time associated with thermal equilibration between steps. In embodiments, the substantially same temperature is maintained within a range that permits efficient hybridization of the first oligonucleotide to the first target polynucleotide, effective cleavage at the cleavable site to produce the first fragment and the second fragment, removal of the fragments, and hybridization of the second oligonucleotide to the second target polynucleotide. The isothermal operation facilitates streamlined workflows for multiplexed detection in biological samples, as the transition between sequential detection rounds does not require heating or cooling cycles that could otherwise introduce delays or affect sample integrity. In embodiments, the isothermal conditions support the use of enzymes and reagents that function optimally within a defined temperature window, thereby maintaining consistent reaction kinetics across the hybridization, cleavage, and detection steps.

In embodiments, the method operates within a temperature range of about 55° C. to about 65° C. This temperature window provides conditions suitable for maintaining oligonucleotide hybridization to target polynucleotides while facilitating efficient removal of cleaved primer fragments. As described above, cleaving the first oligonucleotide at the cleavable site produces a first fragment and a second fragment, wherein each of the first fragment and the second fragment has a melting temperature lower than a melting temperature of the first oligonucleotide. The temperature range of about 55° C. to about 65° C. may be selected such that the intact first oligonucleotide remains stably hybridized to the first target polynucleotide prior to cleavage, while the resulting fragments, having reduced melting temperatures, dissociate and are removed under the same temperature conditions following cleavage. In embodiments, (a), (b), and (c) occur at about 55° C. to about 65° C. In embodiments, the temperature is maintained at about 55° C., about 56° C., about 57° C., about 58° C., about 59° C., about 60° C., about 61° C., about 62° C., about 63° C., about 64° C., or about 65° C. In embodiments, the temperature is maintained at about 58° C. to about 62° C. In embodiments, the temperature is about 60° C. to about 61° C. The selection of a temperature within this range may depend on the specific melting temperatures of the oligonucleotides and their cleaved fragments, as well as the hybridization characteristics of the target polynucleotides. By operating within this temperature range, the method enables sequential rounds of hybridization, detection, cleavage, and removal without requiring substantial temperature changes between steps, thereby simplifying the workflow and reducing potential thermal damage to the cell or tissue sample.

In embodiments, the method operates at about 60° C. Operating at about 60° C. provides a balance between maintaining hybridization stability of intact oligonucleotides and enabling efficient removal of cleaved fragments that have reduced melting temperatures. As described herein, cleaving the first oligonucleotide at the cleavable site produces a first fragment and a second fragment, wherein each of the first fragment and the second fragment has a melting temperature lower than a melting temperature of the first oligonucleotide. At about 60° C., intact oligonucleotides (e.g., sequencing primers) remain stably hybridized to their target polynucleotides, allowing for efficient nucleotide incorporation and detection. Following cleavage, the resulting fragments exhibit melting temperatures that fall below the operating temperature, facilitating their dissociation from the target polynucleotide and subsequent removal during wash steps. In embodiments, (a), (b), and (c) occur at about 60° C. This temperature regime enables the sequential steps of hybridizing the first oligonucleotide to the first target polynucleotide and detecting the first detectable label, cleaving the first oligonucleotide and removing the fragments, and hybridizing the second oligonucleotide to the second target polynucleotide and detecting the second detectable label to proceed without requiring temperature cycling or additional denaturing conditions. The selection of about 60° C. accommodates a range of primer designs, including those with cleavable sites positioned to generate fragments with melting temperatures in the range of about 50° C. to about 58° C., as described in the primer designs herein. Operating at a substantially constant temperature of about 60° C. simplifies workflow integration and reduces the potential for sample damage that may occur with repeated thermal cycling.

In embodiments, the method includes repeating (i), (ii), and (iii) (alternative numbering includes (a), (b), and (c)) in series to detect additional polynucleotides. For example, the method is repeated in series with a second sequencing primer hybridizing to a different polynucleotide, incorporating and detecting a second labeled nucleotide, and again cleaving and removing the primer fragments. Rounds of priming and detection may continue iteratively, enabling the sequential detection of distinct polynucleotides under controlled reaction conditions. In embodiments, the method includes repeating (i), (ii), and (iii) in series with intermediate washing steps to minimize cross-contamination between iterations. In embodiments, the method includes repeating (i), (ii), and (iii) in series a predetermined number of times to detect additional polynucleotides.

In embodiments, the method further includes repeating (a), (b), and (c) in series to detect additional biomolecules. The iterative nature of the method enables multiplexed detection through sequential rounds of primer hybridization, cleavage, and detection. For example, following the detection of a first detectable label associated with a first oligonucleotide hybridized to a first target polynucleotide, the first oligonucleotide may be cleaved at the cleavable site to produce a first fragment and a second fragment, each having a melting temperature lower than the melting temperature of the intact first oligonucleotide. The first fragment and the second fragment may then be removed under standard wash conditions. Subsequently, a second oligonucleotide may be hybridized to a second target polynucleotide, and a second detectable label associated with the second oligonucleotide may be detected. This cycle of hybridization, detection, cleavage, and removal may be repeated with additional oligonucleotides to detect additional biomolecules within the same cell or tissue sample. In embodiments, repeating (a), (b), and (c) in series includes performing 2, 3, 4, 5, 6, 7, 8, 9, 10, or more iterations. In embodiments, repeating (a), (b), and (c) in series includes performing at least 3 iterations. In embodiments, repeating (a), (b), and (c) in series includes performing at least 5 iterations. In embodiments, repeating (a), (b), and (c) in series includes performing 5-10 iterations. In embodiments, repeating (a), (b), and (c) in series includes performing 2-20 iterations. In embodiments, each iteration utilizes a different oligonucleotide that hybridizes to a different target polynucleotide, thereby enabling the detection of a distinct subset of biomolecules in each round. In embodiments, the oligonucleotides used in successive iterations include different primer binding sequences, allowing for selective hybridization to different amplification products or target sequences within the sample. In embodiments, the sequential detection of additional biomolecules occurs at substantially the same temperature across all iterations. In embodiments, each of (a), (b), and (c) occurs at about 55° C. to about 65° C. throughout the iterative process. In embodiments, each of (a), (b), and (c) occurs at about 60° C. throughout the iterative process. Maintaining a substantially constant temperature across iterations simplifies the workflow and reduces the potential for sample degradation or loss of spatial information within the cell or tissue. In embodiments, the iterative detection method enables the identification of 10, 20, 30, 40, 50, 100, or more distinct biomolecules within a single sample. In embodiments, each iteration detects a subset of 2, 3, 4, or more biomolecules based on the incorporation of differently labeled nucleotides. In embodiments, the method includes intermediate washing steps between iterations to remove cleaved primer fragments and residual reagents, thereby minimizing background signal and cross-contamination between successive detection rounds.

In embodiments, the first target polynucleotide and the second target polynucleotide are different polynucleotides. In embodiments, the first target polynucleotide and the second target polynucleotide are amplification products generated by rolling circle amplification. In embodiments, the first oligonucleotide and the second oligonucleotide are sequencing primers. In embodiments, detecting the first detectable label includes incorporating a labeled nucleotide into the first oligonucleotide with a polymerase and detecting the labeled nucleotide. In embodiments, detecting the second detectable label includes incorporating a labeled nucleotide into the second oligonucleotide with a polymerase and detecting the labeled nucleotide.

In embodiments, forming the circular polynucleotide includes ligating a first end and a second end of the probe oligonucleotide together. In embodiments, ligating includes forming a covalent bond from the first end and the second end. As those of skill in the art appreciate, two nucleotide sequences that that are to ligated together will generally directly abut one another.

In embodiments, the method includes contacting a cell or tissue with a polynucleotide probe and hybridizing a first hybridization sequence of the polynucleotide probe to a first target sequence of the RNA or DNA molecule, and hybridizing a second hybridization sequence of the polynucleotide probe to a second target sequence of the RNA or DNA molecule, wherein the RNA or DNA molecule comprises a target sequence between the first target sequence and the second target sequence; extending the polynucleotide probe along the target sequence to generate a complement of the target sequence, and ligating the complement of the target sequence to the polynucleotide probe thereby forming a circular oligonucleotide; extending an amplification primer hybridized to the circular oligonucleotide with a polymerase to generate an extension product comprising the target sequence; and hybridizing a sequencing primer to the extension product and sequencing the target sequence in the cell or tissue, thereby detecting the RNA or DNA molecule. In embodiments, the nucleic acid molecule is cDNA. In embodiments, the nucleic acid molecule is DNA. In embodiments, the nucleic acid molecule is RNA.

In embodiments, the first polynucleotide and the second polynucleotide are formed via rolling circle amplification. For example, circularizable probes, such as padlock probes are employed, wherein each padlock probe comprises a linear oligonucleotide designed to hybridize to distinct target sequences. Upon hybridization, the padlock probe is circularized via a ligase enzyme, thereby forming a closed-loop structure that serves as a template for RCA. In embodiments, RCA is carried out by a polymerase with strand displacement activity, resulting in the synthesis of a concatemeric DNA strand comprising repeated sequences complementary to the target polynucleotide. The RCA products remain spatially localized to their respective target sites. In embodiments, the method includes hybridizing a first sequencing primer to the RCA product of the first polynucleotide, incorporating a labeled nucleotide via a polymerase, and detecting the incorporated labeled nucleotide. Following detection, the first sequencing primer is cleaved into fragments and removed. In embodiments, a second sequencing primer is hybridized to the RCA product of the second polynucleotide, followed by incorporation and detection of a second labeled nucleotide.

Rolling circle amplification (RCA) generates concatemeric amplification products that include multiple tandem copies of the circularized probe sequence. The circularized probe may be formed by ligating the ends of a circularizable probe, such as a padlock probe, following hybridization to a target sequence. The circularized probe serves as a template for the RCA reaction, wherein a strand-displacing polymerase extends an amplification primer around the circular template, producing a long single-stranded DNA molecule containing repeated complementary sequences. In embodiments, the concatemeric amplification products remain spatially localized to the site of probe circularization, preserving the spatial relationship between the amplification product and the original target. The repeated sequence motifs within the concatemer provide multiple binding sites for sequencing primers, enabling detection through sequencing-by-synthesis. In embodiments, the first target polynucleotide includes a concatemeric amplification product generated from a first circularized probe, and the second target polynucleotide includes a concatemeric amplification product generated from a second circularized probe. Each circularized probe may include a distinct primer binding sequence and a barcode sequence, allowing for sequential detection of different targets using different sequencing primers. In embodiments, the RCA reaction is performed using a phi29 polymerase, a Bst large fragment polymerase, or another strand-displacing polymerase. The amplification may be carried out under isothermal conditions, for example at a temperature of about 30° C. to about 42° C., for a duration sufficient to generate amplification products of a desired length. The concatemeric amplification products may include tens to hundreds of copies of the circularized probe sequence, providing signal amplification for subsequent detection steps.

In embodiments, the method further includes ligating the 5′ and 3′ ends of the polynucleotide described herein or the polynucleotide probe described herein to form a circular polynucleotide (i.e., a polynucleotide that is a continuous strand lacking free 5′ and 3′ ends). In embodiments, the method includes ligating the 5′ and 3′ ends of the polynucleotide to form a circular polynucleotide, wherein the circular polynucleotide includes the target nucleic acid. In embodiments, the method includes ligating the 5′ and 3′ ends of the polynucleotide to form a circular polynucleotide, wherein the circular polynucleotide includes the oligonucleotide barcode. In embodiments, the ligation includes enzymatic ligation. In embodiments, ligating includes enzymatic ligation including a ligation enzyme (e.g., CircLigase™ enzyme, Taq DNA Ligase, HiFi Taq DNA Ligase, T4 ligase, PBCV-1 DNA Ligase (also known as SplintR® ligase) or Ampligase™ DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR® ligase) or a Taq DNA Ligase. In embodiments, the ligase enzyme is a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR® ligase), and combinations thereof. In embodiments, prior to ligating the 5′ and 3′ ends of the polynucleotide probe to form a circular polynucleotide, the method includes extending the 3′ end of the polynucleotide probe (using a polymerase to incorporate one or more nucleotides) along the oligonucleotide to generate a complementary sequence and ligating the extended 3′ end of the polynucleotide probe to the 5′ end of the polynucleotide probe.

In embodiments, the oligonucleotide is covalently attached to a protein-specific binding agent, wherein the protein-specific binding agent is an antibody, single-chain Fv fragment (scFv), affimer, aptamer, single-domain antibody (sdAb), or antibody fragment-antigen binding (Fab). In embodiments, the oligonucleotide is covalently attached to an antibody or single-domain antibody (sdAb). In embodiments, the oligonucleotide is covalently attached to an antibody. In embodiments, the oligonucleotide is covalently attached to a single-chain Fv fragment (scFv). In embodiments, the oligonucleotide is covalently attached to an affimer. In embodiments, the oligonucleotide is covalently attached to an aptamer. In embodiments, the oligonucleotide is covalently attached to a single-domain antibody (sdAb). In embodiments, the oligonucleotide is covalently attached to an antibody fragment-antigen binding (Fab). In embodiments, the oligonucleotide is covalently attached to an antibody or single-domain antibody (sdAb). The design and preparation of protein-specific binding agent oligonucleotide conjugates is known, for example various different binding moieties which may be used, the design of probe oligonucleotides, and the coupling of such oligonucleotides to the binding moieties to form the conjugates. The details and principles may be applied to the design of the probes for use in the methods described herein. For example, reference may be made to WO 2007/107743, U.S. Pat. Nos. 7,306,904 and 6,878,515 which are incorporated herein by reference.

In embodiments, the polynucleotide (e.g., the first polynucleotide and/or the second polynucleotide) is attached to a specific binding agent (e.g., an antibody) via a linker (e.g., a bioconjugate linker). In embodiments, the polynucleotide is attached to the protein-specific binding agent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety). For example, polynucleotide may be covalently attached using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to polynucleotide) with an avidin or streptavidin linker. In embodiments, the polynucleotide includes a barcode, wherein the barcode is a known sequence associated with the specific binding reagent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, the first polynucleotide is covalently attached to a first specific binding agent and the second polynucleotide is covalently attached to a second specific binding agent, wherein the first specific binding agent and the second specific binding agent are each independently an antibody, single-chain Fv fragment (scFv), antibody fragment-antigen binding (Fab), affimer, or an aptamer. In embodiments, the first polynucleotide and the second polynucleotide act as primers and are bound to a specific binding agent (e.g., antibody). In embodiments, following hybridization of a padlock probe to a target nucleic acid sequence of the primer, the padlock probe undergoes circularization via a ligase enzyme. The 3′ end of the first or second polynucleotide, acting as a primer, is then extended by a polymerase, resulting in the synthesis of a rolling circle amplification (RCA) product, alternatively referred to as the first or second polynucleotide. In embodiments, the RCA product comprises a concatemeric DNA strand containing repeated sequences complementary to the padlock probe. In embodiments, the method includes hybridizing a sequencing primer to the RCA product of the first polynucleotide, incorporating a labeled nucleotide via a polymerase, and detecting the incorporated labeled nucleotide. Following detection, the first sequencing primer is cleaved into fragments and removed. In embodiments, a second sequencing primer is hybridized to the RCA product of the second polynucleotide, followed by incorporation and detection of a second labeled nucleotide.

In embodiments, the antibody-oligonucleotide (Ab-O) conjugate includes an antibody covalently linked to an oligonucleotide through a linker moiety. The linker moiety may be attached to the antibody at various positions, including lysine residues, cysteine residues, or engineered conjugation sites on the antibody. In embodiments, the oligonucleotide is conjugated to the antibody via a heterobifunctional crosslinker, such as a maleimide-thiol linkage, an NHS ester-amine linkage, or a click chemistry-based linkage. In embodiments, the oligonucleotide portion of the Ab-O conjugate includes a unique barcode sequence that identifies the antibody and the target protein to which the antibody binds. In embodiments, the oligonucleotide portion includes a hybridization sequence complementary to a portion of the circularizable probe, enabling the circularizable probe to bind to the Ab-O conjugate at the site of the target protein. In embodiments, the Ab-O conjugate is prepared by reacting a functionalized antibody with a functionalized oligonucleotide under conditions that promote covalent bond formation while preserving the antigen-binding activity of the antibody. In embodiments, the Ab-O conjugate retains specific binding affinity for its target protein with a dissociation constant (KD) of less than 10⁻⁷ M, less than 10⁻⁸ M, or less than 10⁻⁹ M. In embodiments, the oligonucleotide portion of the Ab-O conjugate has a length of 20 to 100 nucleotides, 30 to 80 nucleotides, or 40 to 60 nucleotides. In embodiments, the Ab-O conjugate includes a monoclonal antibody, a polyclonal antibody, or a recombinant antibody.

Padlock probes (e.g., circularizable oligonucleotides, also referred to as circularizable probes) are specialized ligation probes, examples of which are known in the art, see for example Nilsson M, et al. Science. 1994; 265(5181):2085-2088), and has been applied to detect transcribed RNA in cells, see for example Christian A T, et al. Proc Natl Acad. Sci USA. 2001; 98(25):14238-14243, both of which are incorporated herein by reference in their entireties. In embodiments, the padlock probe is approximately 50 to 200 nucleotides. In embodiments, a padlock probe has a first domain that is capable of hybridizing to a first target sequence domain, and a second ligation domain, capable of hybridizing to an adjacent second sequence domain. The configuration of the padlock probe is such that upon ligation of the first and second ligation domains of the padlock probe, the probe forms a circular polynucleotide, and forms a complex with the sequence (i.e., the sequence it hybridized to, the target sequence) wherein the target sequence is “inserted” into the loop of the circle. Padlock probes are useful for the methods provided herein and include, for example, padlock probes for genomic analyses, as exemplified by Gore, A. et al. Nature 471, 63-67 (2011); Porreca, G. J. et al. Nat Methods 4, 931-936 (2007); Li, J. B. et al. Genome Res 19, 1606-1615 (2009), Zhang, K. et al. Nat Methods 6, 613-618 (2009); Noggle, S. et al. Nature 478, 70-75 (2011); and Li, J. B. et al. Science 324, 1210-1213 (2009), the content of each of which are incorporated by reference in their entirety.

In embodiments, the polynucleotide probe described herein is a circularizable probe. In embodiments, the circularizable probe (e.g., the circularizable oligonucleotide) comprises a 5′ end and a 3′ end, wherein a first region at the 5′ end is complementary to a first sequence of a target polynucleotide, and wherein a second region at the 3′ end is complementary to a second sequence of the target polynucleotide. In embodiments, the first sequence and the second sequence of the target polynucleotide are adjacent to each other. In embodiments, the first sequence and the second sequence of the target polynucleotide are separated by 1 or more nucleotides. In embodiments, the first sequence and the second sequence of the target polynucleotide are separated by 1, 5, 10, 20, 30, 40, 50, 75, 100, or more nucleotides. In embodiments, the first sequence and the second sequence of the target polynucleotide flank a target sequence. In embodiments, the target sequence is a barcode sequence. In embodiments, the gap sequence is 5, 10, 15, 20, 25, 30, 40, 50, 60, 70, 80, 90, 100, 125, 150, 175, or 200 nucleotides. In embodiments, the gap sequence is 5 to 150 nucleotides. In embodiments, the gap sequence is 1, 2, 3, 4, or 5 nucleotides.

In embodiments, the circularized probe includes a barcode sequence. As described previously, the circularizable probe may be hybridized to an oligonucleotide conjugated to a specific binding agent, wherein the specific binding agent is an antibody, single-chain Fv fragment (scFv), antibody fragment-antigen binding (Fab), affimer, or an aptamer. The barcode sequence included in the circularized probe enables identification of the target protein through sequencing-based detection. In embodiments, the barcode sequence is a known nucleotide sequence that is associated with a particular specific binding agent, such that detecting and decoding the barcode sequence identifies the specific binding agent and, consequently, the target protein to which the specific binding agent is bound. In embodiments, the barcode sequence is positioned within the circularized probe such that upon rolling circle amplification, the amplification product includes multiple copies of the barcode sequence arranged in tandem. In embodiments, sequencing the barcode sequence includes hybridizing a sequencing primer to a primer binding sequence within the amplification product and incorporating labeled nucleotides to decode the barcode sequence. In embodiments, the barcode sequence is selected from a known set of barcode sequences, wherein each barcode sequence in the known set is associated with a different specific binding agent. In embodiments, the barcode sequence has a length of about 5 to about 20 nucleotides. In embodiments, the barcode sequence has a length of about 8 to about 15 nucleotides. In embodiments, the barcode sequence has a length of about 10 nucleotides. In embodiments, the barcode sequences in the known set of barcode sequences are distinguished by a Hamming distance of at least 3, at least 4, or at least 5 nucleotide positions, thereby enabling error detection and correction during sequencing-based identification of the target protein.

In embodiments, the first polynucleotide and the second polynucleotide are in a tissue section. In embodiments, the tissue section comprises a biological sample derived from a subject, including but not limited to, formalin-fixed paraffin-embedded (FFPE) tissues, fresh-frozen tissues, cryosectioned tissues, or microdissected cellular regions. In embodiments, the tissue section includes heterogeneous cell populations, such as epithelial cells, stromal cells, immune cells, or neurons, enabling spatially resolved detection of nucleic acids within specific microenvironments. In embodiments, the tissue section retains structural integrity, preserving cellular morphology and spatial relationships among biomolecules. In embodiments, the tissue section is subjected to permeabilization or antigen retrieval treatments to facilitate probe accessibility while maintaining spatial context. In embodiments, the tissue section is mounted on a solid support, such as a glass slide or a solid support. In embodiments, the first polynucleotide and the second polynucleotide are in the same tissue section.

In embodiments, the first sequencing primer includes one or more cleavable sites. In embodiments, the cleavable site includes a deoxyuracil triphosphate (dUTP), a deoxy-8-oxo-guanine triphosphate (d-8-oxoG), a methylated nucleotide, or a ribonucleotide. In embodiments, the cleavable site includes a chemically or enzymatically labile bond that enables controlled cleavage of the sequencing primer following nucleotide incorporation and detection. In embodiments, cleavage of the site is facilitated by enzymatic digestion, such as uracil-DNA glycosylase (UDG) cleavage of dUTP-containing primers or RNase hydrolysis of ribonucleotide-containing primers. In embodiments, the cleavable site is susceptible to chemical cleavage, such as treatment with acidic or oxidative agents that selectively degrade modified nucleotides.

In embodiments, the first oligonucleotide includes two or more cleavable sites. In embodiments, the first oligonucleotide includes a C6-S—S linker. In embodiments, the first oligonucleotide includes a sequence described herein, for example, in Table 3. In embodiments, the first oligonucleotide includes a first sequence described in Table 3 and a second sequence described in Table 3. In embodiments, the cleavable site of the first oligonucleotide includes a modified nucleotide that may be cleaved enzymatically. In embodiments, the cleavable site includes a deoxyuracil triphosphate (dUTP). Deoxyuracil triphosphate may be incorporated into an oligonucleotide during synthesis and subsequently cleaved by treatment with uracil DNA glycosylase (UDG), which removes the uracil base to generate an abasic site. The abasic site may then be cleaved by an endonuclease, such as endonuclease VIII or apurinic/apyrimidinic endonuclease, to fragment the oligonucleotide at the cleavable site. In embodiments, the cleavable site includes a deoxy-8-oxo-guanine triphosphate (d-8-oxoG). Deoxy-8-oxo-guanine is an oxidized form of guanine that may be recognized and excised by Formamidopyrimidine DNA Glycosylase (Fpg), also known as 8-oxoguanine DNA glycosylase. Fpg removes the 8-oxoguanine base and cleaves the phosphodiester backbone at the resulting abasic site, thereby fragmenting the oligonucleotide. In embodiments, the cleavable site includes a methylated nucleotide. Methylated nucleotides, such as 5-methylcytosine, may be recognized by methylation-sensitive restriction enzymes or by glycosylases that specifically excise methylated bases. Cleavage at a methylated nucleotide site may be achieved through enzymatic recognition and excision of the methylated base, followed by backbone cleavage at the resulting abasic site. In embodiments, the cleavable site includes a ribonucleotide. A ribonucleotide incorporated into an otherwise deoxyribonucleotide oligonucleotide may be cleaved by an endoribonuclease, such as RNase H or RNase HII, when the oligonucleotide is hybridized to a complementary DNA strand. RNase H specifically cleaves the RNA portion of an RNA-DNA hybrid, generating a nick or gap at the ribonucleotide position. In embodiments, the cleavable site includes one or more ribonucleotides, and cleavage may be performed under alkaline conditions or by enzymatic treatment to fragment the oligonucleotide. As described herein, cleavage at the cleavable site produces a first fragment and a second fragment, wherein each of the first fragment and the second fragment has a melting temperature lower than a melting temperature of the intact first oligonucleotide.

In embodiments endoribonuclease enzymes may be used to specifically cleave primers having a ribonucleotide (RNA) position when the primer is hybridized to polynucleotide. In embodiments, the endoribonuclease is an RNAse H, such as RNase HII. In embodiments, the endoribonuclease is a thermostable enzyme or a thermophilic, hotstart enzyme (e.g., a thermostable RNase HII enzyme and a thermophilic, hotstart RNaseHII enzyme). In embodiments, chemical cleavage of the sequencing primer includes incubating the sample with a reducing agent, such as tris(2-carboxyethyl)phosphine (TCEP), followed by washing of the sample in phosphate buffered saline comprising 0.1 Triton-X 100 (PBT) and/or phosphate buffered saline (PBS). In embodiments, the cleavable site includes a chemically cleavable linker. In embodiments, the cleavable site includes a disulfide linker. In embodiments, the sequencing primer includes carbon spacers. Carbon spacers may include spacers that may be 3 to 36 carbon atoms in length. Common examples of internal oligonucleotide carbon spacers include spacers that are 3, 9, and 18 carbon atoms in length. Carbon spacers may also be used to adjust the melting temperature (Tm) of the primer. In embodiments, the first sequencing primer includes a C6-S—S linker (alternatively referred to as a SS—C6 linker). For example, a C6-S—S linker has the formula:

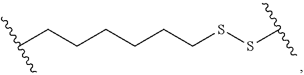 [SMILES: *CCCCCCSS*]

which connects the two sequences. In embodiments, the first sequencing primer includes a C6-S—S—C6 linker, for example, having the formula:

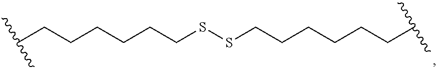 [SMILES: *CCCCCCSSCCCCCC*]

which connects the two sequences. In embodiments, the first sequencing primer includes a diol.

In embodiments, the cleavable site includes a disulfide scissile internucleosidic linkage. A disulfide scissile internucleosidic linkage refers to a sulfur-sulfur bond positioned within the backbone of an oligonucleotide that can be selectively cleaved under reducing conditions. The disulfide bond provides a chemically labile site that remains stable during hybridization and sequencing reactions but undergoes cleavage upon exposure to a reducing agent, thereby fragmenting the oligonucleotide into two or more segments. In embodiments, the disulfide scissile internucleosidic linkage includes a C6-S—S linker, which incorporates a six-carbon spacer flanking the disulfide bond. The C6-S—S linker may have the formula

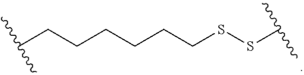 [SMILES: *CCCCCCSS*]

In embodiments, the disulfide scissile internucleosidic linkage includes a C6-S—S—C6 linker, which incorporates six-carbon spacers on both sides of the disulfide bond, for example having the formula

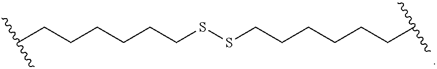 [SMILES: *CCCCCCSSCCCCCC*]

The carbon spacers provide flexibility within the oligonucleotide structure and may be used to adjust the positioning of the cleavable site relative to the hybridized region of the primer. In embodiments, cleaving the disulfide scissile internucleosidic linkage includes contacting the oligonucleotide with a reducing agent. Suitable reducing agents include tris(2-carboxyethyl)phosphine (TCEP), tris(3-hydroxypropyl)phosphine (THPP), dithiothreitol (DTT), or other thiol-containing compounds capable of reducing disulfide bonds. Upon cleavage of the disulfide bond, the oligonucleotide is fragmented into smaller segments, each having a lower melting temperature than the intact oligonucleotide. The reduction in melting temperature facilitates dissociation of the cleaved fragments from the target polynucleotide under standard wash conditions, enabling efficient primer removal without requiring elevated temperatures or harsh denaturing agents. In embodiments, the cleavable site of the first oligonucleotide includes a disulfide bond, a deoxyuracil triphosphate (dUTP), a deoxy-8-oxo-guanine triphosphate (d-8-oxoG), a methylated nucleotide, or a ribonucleotide. In embodiments, cleaving the first oligonucleotide includes contacting the first oligonucleotide with a reducing agent, a nuclease, or a glycosylase. In embodiments, the reducing agent includes tris(2-carboxyethyl)phosphine (TCEP) or tris(3-hydroxypropyl)phosphine (THPP).

In embodiments, cleaving the first sequencing primer includes contacting the first sequencing primer with a reducing agent, sodium periodate, or a nuclease. In embodiments, cleaving the first sequencing primer includes contacting the first sequencing primer with RNase, Formamidopyrimidine DNA Glycosylase (Fpg), a restriction enzyme, or uracil DNA glycosylase (UDG). Other methods of cleavage can be used as an alternative to restriction enzymes or nicking enzymes, including chemical cleavage (e.g. cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker. Any suitable enzymatic, chemical, or photochemical cleavage reaction may be used to cleave the cleavage site. The cleavage reaction may result in removal of a part or the whole of the polynucleotide primer being cleaved. Suitable cleavage means include, for example, restriction enzyme digestion, in which case the cleavage site is an appropriate restriction site for the enzyme which directs cleavage of one or both strands of a duplex template; RNase digestion or chemical cleavage of a bond between a deoxyribonucleotide and a ribonucleotide, in which case the cleavage site may include one or more ribonucleotides; chemical reduction of a disulfide linkage with a reducing agent (e.g., THPP or TCEP), in which case the cleavage site should include an appropriate disulfide linkage; chemical cleavage of a diol linkage with periodate, in which case the cleavage site should include a diol linkage; generation of an abasic site and subsequent hydrolysis, etc. In embodiments, the cleavage site is included in the surface immobilized primer (e.g., within the polynucleotide sequence of the primer). In embodiments, the sequencing primer may include a diol linkage which permits cleavage by treatment with periodate (e.g., sodium periodate). It will be appreciated that more than one diol can be included at the cleavage site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine.

In embodiments, the cleavable site includes one or more deoxyuracil triphosphates (dUTPs), deoxy-8-oxo-guanine triphosphates (d-8-oxoGs), methylated nucleotides, or ribonucleotides. In embodiments, the cleavable site includes one or more deoxyuracil triphosphates (dUTPs). In embodiments, the cleavable site includes one or more deoxy-8-oxo-guanine triphosphates (d-8-oxoGs). In embodiments, the cleavable site includes one or more methylated nucleotides. In embodiments, the cleavable site includes one or more ribonucleotides. The one or more cleavable sites may include a modified nucleotide, ribonucleotide, or a sequence containing a modified or unmodified nucleotide that is specifically recognized by a cleavage agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes, and include modified ribonucleotides and deoxyribonucleotides including abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. In embodiments, the cleavable site includes an abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C.

In embodiments, the cleavable site is cleaved by contacting the oligonucleotide with a cleaving agent. In embodiments, the cleavable site can be cleaved by enzymes, nucleophilic/basic reagents, reducing agents, photo-irradiation, electrophilic/acidic reagents, organometallic and metal reagents, or oxidizing reagents. In embodiments, the cleavable site can be chemically cleaved by a chemical. In embodiments, the chemically cleavable site is split in response to the presence of a acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, a chemically cleavable linker is non-enzymatically cleavable. In embodiments, cleaving includes removing. In embodiments, the cleavable linker includes one or more cleavable site(s). Any suitable enzymatic, chemical, or photochemical cleavage reaction may be used to cleave the cleavable site. In embodiments, cleaving the cleavable linker can be chemical cleavage of a bond between a deoxyribonucleotide and a ribonucleotide, in which case, the cleavable site may include one or more ribonucleotides. In embodiments, cleaving of the cleavable site can be a chemical reduction of a disulfide linkage with a reducing agent (e.g., THPP or TCEP), in which case, the cleavable site should include an appropriate disulfide linkage; chemical cleavage of a diol linkage with periodate, in which case, the cleavable site should include a diol linkage; generation of an abasic site and subsequent hydrolysis, etc. In embodiments, the linker includes a diol linkage, which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, the oligonucleotide includes two or more cleavable sites. Any suitable enzymatic, chemical, or photochemical cleavage reaction may be used to cleave the cleavable site. In embodiments, the cleavable site includes one or more deoxyuracil nucleobases (dUs). In embodiments, the cleavable site includes multiple deoxyuracil nucleobases (dUs). In embodiments, the cleavable site includes a plurality of consecutive nucleobases (dUs). In embodiments, the cleavable site is cleaved as a result of enzymatic cleaving. In embodiments, the cleaving agent is an enzyme. In embodiments, the enzyme is one or more restriction enzymes. The restriction enzyme will recognize a particular restriction site sequences in one or both strands of the cleavable site, resulting in cleavage of the cleavable site. The resulting restriction enzyme digestion may cleave one or both strands of a duplex template. The enzymatic cleavage reaction may result in removal of a part or the whole of the strand being cleaved. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, cleaving includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage is performed with about 1 unit (U) to about 50 U of restriction endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of restriction endonuclease. In embodiments, cleavage is performed with about 5 U to about 10 U of restriction endonuclease. In embodiments, cleavage is performed with about 10 U to about 15 U of restriction endonuclease. In embodiments, cleavage is performed with about 15 U to about 20 U of restriction endonuclease. In embodiments, cleavage is performed with about 20 U to about 25 U of restriction endonuclease. In embodiments, cleavage is performed with about 25 U to about 35 U of restriction endonuclease. In embodiments, cleavage is performed with about 35 U to about 50 U of restriction endonuclease. In embodiments, cleavage is performed with about 1, 2, 3, 4, 5, 6, 7, 8, 9, 10, 11, 12, 13, 14, 15, 16, 17, 18, 19, 20, 21, 22, 23, 24, 25, 30, 35, 40, 45 or 50 U of restriction endonuclease. In embodiments, cleavage is performed with less than about 1 U of restriction endonuclease. In embodiments, cleavage is performed with greater than about 50 U of restriction endonuclease.

In embodiments, the first polynucleotide includes a barcode associated with a biomolecule, and the method further includes sequencing the barcode. In embodiments, the methods described herein further includes sequencing in situ. In embodiments, the method includes incorporating a target sequence into a circular polynucleotide, amplifying the circular polynucleotide (e.g., amplifying the circular polynucleotide via rolling circle amplification) to generate an amplification product, and sequencing the amplification product. In embodiments, sequencing the amplification product includes identifying or determining the target sequence (or a complement thereof). Known techniques for incorporating a target sequence into a circular polynucleotide include methods and compositions described, for example, in U.S. Pat. Nos. 11,434,525, 11,680,288, 11,753,678, and 12,006,534, each of which are incorporated herein by reference.

In embodiments, the method includes detecting a plurality of different targets. The selection of suitable targets is influenced by the type of analysis required. In some embodiments, a target may indicate the presence or absence of an analyte, while in other embodiments, a target may provide information regarding the state of the biological sample. In the context of tissue-based assays, the disclosed methods may facilitate the differentiation of cell types, the comparison of developmental stages, the detection of disease markers, or the classification of pathological conditions. The disclosed methods allow for the detection of a variety of biomolecules, including peptides, proteins such as antibodies, affibodies, or aptamers, nucleic acids such as DNA, RNA, polynucleotides, or aptamers, and polysaccharides such as lectins or sugars. Additional biomolecules that may be detected include lipids, enzymes, enzyme substrates, ligands, receptors, antigens, and haptens. In some embodiments, the detected targets may predominantly include proteins or nucleic acids. Some targets may be characteristic of particular cell types, while others may be associated with specific diseases or conditions. A target may be present on the surface of a biological sample, such as an antigen within a tissue section, or may exist in solution, such as an antibody in a buffer or body fluid. In some instances, a target may not be inherently accessible on the surface of a biological sample and may require processing steps, including antigen retrieval, enzymatic digestion, epitope unmasking, or blocking, to facilitate detection. Targets may also be present in body fluids such as blood, plasma, serum, or urine, or may be localized within a tissue, either at the cell surface or within the intracellular compartment.

In some embodiments, the methods described herein may be applied to the detection of prognostic markers, hormone receptors, lymphoid markers, tumor markers, cell cycle regulators, neural tissue and tumor markers, and cluster differentiation antigens. Prognostic markers may include enzymatic targets such as galactosyl transferase II, neuron-specific enolase, proton ATPase-2, and acid phosphatase. Hormone-related targets may include human chorionic gonadotropin, adrenocorticotropic hormone, carcinoembryonic antigen, prostate-specific antigen, estrogen receptor, progesterone receptor, androgen receptor, thyroid hormone receptor, and insulin receptor. Lymphoid markers may include B cell and T cell antigens, immunoglobulin subtypes, HLA class I and II antigens, granzyme B, and secretory leukocyte protease inhibitors. Tumor markers may include alpha-fetoprotein, tumor-associated mucin antigens, epithelial membrane antigen, matrix metalloproteinases, melanoma antigens, prostate-specific antigen, and tyrosinase. Cell cycle regulators may include members of the Bcl family, caspases, cyclins, CDK inhibitors, and topoisomerases. Neural tissue and tumor markers may include neurofilaments, beta-amyloid, synaptic proteins, and tyrosine hydroxylase. Cluster differentiation antigens may include T cell markers, B cell markers, myeloid lineage markers, adhesion molecules, activation markers, hematopoietic stem cell markers, and apoptosis-related markers.

The methods described herein provide an approach for detecting biomolecules across a range of biological applications, including disease diagnostics, biomarker discovery, and molecular profiling. The ability to detect a diverse set of targets supports applications in biomedical research and personalized medicine. The detection of such targets may be achieved through sequencing-based approaches, antibody-oligonucleotide conjugates, rolling circle amplification, or hybridization-based methods, among others. In some embodiments, multiplexed detection strategies may be employed to analyze multiple targets within a single sample, enabling a comprehensive assessment of biomolecular states within a biological system.

In embodiments, the first polynucleotide and the second polynucleotide are in a cell. In embodiments, the first polynucleotide and the second polynucleotide are in a cell which forms part of a tissue. In embodiments, the first polynucleotide and the second polynucleotide are in a tissue section. In embodiments, the tissue section may be first fixed and then dehydrated through an ascending series of alcohols, infiltrated and embedded with paraffin or other sectioning media so that the tissue sample may be sectioned. In embodiments, a tissue sample may be sectioned and subsequently fixed. In some embodiments, the tissue sample may be embedded and processed in paraffin. Examples of paraffin that may be used include, but are not limited to, Paraplast, Broloid, and Tissuemay. Once the tissue sample is embedded, the sample may be sectioned by a microtome into sections that may have a thickness in a range of from about three microns to about five microns. Once sectioned, the sections may be attached to a solid support (e.g., a slide or a flow cell). In embodiments, if paraffin is used as the embedding material, the tissue sections may be deparaffinized and rehydrated in water. The tissue sections may be deparaffinized, for example, by using organic agents (such as, xylenes or gradually descending series of alcohols).

In embodiments, a series of probes may be contacted with the biological sample in a sequential manner to obtain a multiplexed analysis of the biological sample. In some embodiments, a series of primer sets (e.g., including 2-4 primers in one set) may be contacted with the biological sample in a sequential manner to obtain a multiplexed analysis of the biological sample. Multiplexed analysis generally refers to analysis of multiple targets in a biological sample using the same detection mechanism.

A morphological stain may be contacted with a biological sample at various stages of the disclosed methods, including before, during, or after any of the aforementioned steps. In embodiments, a morphological stain may be introduced during the initial primer contact step, allowing for simultaneous staining and target detection. In embodiments, a morphological stain may be applied after sequencing. Where morphological stains may introduce background noise that interferes with the fluorescent signal generated by the labeled nucleotide, the stain may be applied only after the sequencing is completed. For example, morphological stains such as hematoxylin and eosin (H&E) may be sequentially imaged and registered after the completion of the disclosed methods to preserve signal clarity. In some embodiments, chromophores, fluorophores, or enzyme/enzyme substrates may be used as morphological stains. Suitable chromophores for morphological staining may include eosin for staining alkaline cellular components such as the cytoplasm, hematoxylin for nucleic acids, Orange G for red blood cells and certain glandular tissues, and Light Green SF for collagen. Other chromophores that may be used for morphological staining include Romanowsky-Giemsa for overall cell morphology, May-Grunwald for blood cell differentiation, Feulgen reagent for DNA, Alcian blue for carbohydrates, Sudan Black for lipids, and various trichrome stains for distinguishing muscle fibers, cytoplasm, and connective tissues. Fluorescent morphological stains may also be used for visualization of cellular structures. Examples of suitable fluorescent stains include 4′,6-diamidino-2-phenylindole (DAPI) for nucleic acids, Hoechst 33258 and Hoechst 33342 for DNA staining, and propidium iodide for nucleic acid visualization. Other fluorescent stains may include acridine orange for DNA and RNA, fluorescein-phalloidin for actin fibers, and ethidium bromide for nucleic acids. High-affinity DNA fluorophores such as POPO, BOBO, YOYO, and TOTO, as well as fluorescent protein fusions to DNA-binding proteins such as histones, may also be employed. The selection and application of a morphological stain may be adapted based on the specific requirements of the biological sample and the analytical approach used in the disclosed methods. In some embodiments, the morphological stain may be selected to enhance contrast while minimizing interference with other detection signals. The disclosed methods accommodate a wide range of morphological stains, allowing for structural and molecular analyses to be conducted within the same sample.

In embodiments, cleaving the sequencing primer (e.g., the first or second sequencing primer) produces fragments with distinguishable melting temperatures (e.g., melting temperatures that differ by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° C. from one another.

In embodiments, the method may further include performing an amplification step to amplify the polynucleotides. For example, the amplification can be isothermal amplification or one or more polymerase chain reaction cycles. Isothermal amplification techniques include, for example, strand displacement amplification (SDA), loop-mediated amplification (LAMP), rolling circle amplification (RCA), and helicase-dependent amplification (HAD). In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the method includes contacting the target with an amplification primer and oligonucleotide primer in the same reaction (e.g., simultaneously). In embodiments, the method includes fixing the amplification products (e.g., contacting the amplification product with formalin).

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g., a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold and milieu. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, amplifying includes incubating the circular polynucleotide with a strand-displacing polymerase for about 15 minutes to about 2 hours. In embodiments, amplifying includes incubating the circular polynucleotide with a strand-displacing polymerase for about 30 minutes to about 60 minutes. In embodiments, amplifying includes binding an amplification primer to the primer binding sequence and extending the amplification primer with a strand-displacing polymerase.

In embodiments, amplifying the circular oligonucleotide includes incubating the circular oligonucleotide with the strand-displacing polymerase (a) for about 1 minute to about 2 hours, and/or (b) at a temperature of about 20° C. to about 50° C. In embodiments, amplifying the circular oligonucleotide includes incubating the circular oligonucleotide with the strand-displacing polymerase for about 1 minute to about 2 hours. In embodiments, amplifying the circular oligonucleotide includes incubating the circular oligonucleotide with the strand-displacing polymerase for about 5, about 10, about 20, about 30, about 40, about 45, about 50, about 55, or about 60 minutes. In embodiments, amplifying the circular oligonucleotide includes incubating the circular oligonucleotide with the strand-displacing polymerase for about 5 minutes. In embodiments, amplifying the circular oligonucleotide includes incubating the circular oligonucleotide with the strand-displacing polymerase for about 10 minutes. In embodiments, amplifying the circular oligonucleotide includes incubating the circular oligonucleotide with the strand-displacing polymerase for about 20 minutes. In embodiments, amplifying the circular oligonucleotide includes incubating the circular oligonucleotide with the strand-displacing polymerase for about 30 minutes. In embodiments, amplifying the circular oligonucleotide includes incubating the circular oligonucleotide with the strand-displacing polymerase for about 45 minutes. In embodiments, amplifying the circular oligonucleotide includes incubating the circular oligonucleotide with the strand-displacing polymerase for about 60 minutes.

In embodiments, amplifying the circular oligonucleotide includes incubating the circular oligonucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular oligonucleotide includes incubating the circular oligonucleotide with the strand-displacing polymerase for about 1, about 2, about 3, about 4, about 5, about 6, about 7, about 8, about 9, about 10, about 11, about 12 hours, about 13 hours, about 14 hours, about 15 hours, about 16 hours, about 17 hours, about 18 hours, about 19, hours, or about 20 hours. In embodiments, amplifying the circular oligonucleotide includes incubating the circular oligonucleotide with the strand-displacing polymerase for more than 12 hours.

In embodiments, amplifying includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, amplifying includes incubation in a buffer at 30° C. to 40° C. In embodiments, amplifying includes incubation in a buffer at 40° C. to 50° C. In embodiments, amplifying the circular oligonucleotide includes incubating the circular oligonucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. In embodiments, amplifying includes rolling circle amplification for 15 minutes to 16 hours. In embodiments, amplifying includes rolling circle amplification for 15 minutes to 4 hours. In embodiments, amplifying includes rolling circle amplification for 15 minutes to 2 hours. In embodiments, amplifying includes rolling circle amplification for 15 minutes to 1 hour.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput.

In embodiments, the first detectable label is a fluorescent label covalently attached to the first oligonucleotide. The covalent attachment of the fluorescent label to the first oligonucleotide enables direct detection through fluorescence imaging without requiring additional labeling steps or secondary detection reagents. The fluorescent label may be attached to the first oligonucleotide at various positions, including the 5′ terminus, the 3′ terminus, or at an internal nucleotide position. In embodiments, the fluorescent label is attached to the first oligonucleotide via a linker moiety, wherein the linker moiety may be a cleavable linker or a non-cleavable linker. The covalent attachment may be formed through standard oligonucleotide synthesis methods or through post-synthetic conjugation reactions involving bioconjugate reactive moieties as described herein.

In embodiments, the fluorescent label includes a fluorophore selected from xanthene dyes, cyanine dyes, coumarin dyes, rhodamine dyes, or derivatives thereof. In embodiments, the fluorescent label is an Alexa Fluor® dye, a Cy® dye, a DyLight® dye, an Atto® dye, or a CF® dye. The selection of the fluorescent label may be based on the excitation and emission wavelengths compatible with the imaging system, the photostability of the fluorophore, and the spectral separation from other fluorescent labels used in multiplexed detection assays. In embodiments, the fluorescently labeled oligonucleotide is detected using fluorescence microscopy, confocal microscopy, or other fluorescence-based imaging modalities. The fluorescent signal emitted by the fluorescent label upon excitation provides spatial information regarding the location of the first oligonucleotide hybridized to the first target polynucleotide within the cell or tissue. In embodiments, the intensity of the fluorescent signal correlates with the abundance of the first target polynucleotide, enabling quantitative analysis of biomolecule expression levels.

In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP) into the first sequencing primer.

In embodiments, the method includes incorporating a plurality of labeled nucleotides into the first oligonucleotide in a plurality of sequencing cycles prior to cleaving the first oligonucleotide at the cleavable site. As described above, the first oligonucleotide may be a sequencing primer, and detecting the first detectable label may include incorporating a labeled nucleotide into the first oligonucleotide with a polymerase and detecting the labeled nucleotide. In embodiments, multiple sequencing cycles are performed using the first oligonucleotide before the cleavage and removal steps occur, thereby enabling the reading of multiple bases from a single primer hybridization event. In embodiments, each sequencing cycle includes extending the first oligonucleotide by incorporating a single labeled nucleotide, detecting the incorporated labeled nucleotide, and preparing the extended oligonucleotide for a subsequent incorporation event. In embodiments, the labeled nucleotide includes a reversible terminator moiety that prevents further extension until the terminator moiety is removed. Following detection of the incorporated labeled nucleotide, the reversible terminator moiety may be cleaved to regenerate a free 3-hydroxyl group, thereby permitting incorporation of an additional labeled nucleotide in a subsequent sequencing cycle.

In embodiments, the first oligonucleotide is a sequencing primer and detecting the first detectable label includes binding a labeled nucleotide to the first oligonucleotide with a polymerase and detecting the labeled nucleotide. In embodiments, the first oligonucleotide is a sequencing primer and detecting the first detectable label includes incorporating a labeled nucleotide into the first oligonucleotide with a polymerase and detecting the labeled nucleotide. In embodiments, the method includes incorporating a plurality of labeled nucleotides into the first oligonucleotide in a plurality of sequencing cycles.

In embodiments, the plurality of sequencing cycles includes 2, 3, 4, 5, 6, 7, 8, 9, 10, 15, 20, 25, 30, 40, 50, or more sequencing cycles performed prior to cleaving the first oligonucleotide. In embodiments, the plurality of sequencing cycles includes 5 to 20 sequencing cycles. In embodiments, the plurality of sequencing cycles includes 10 to 50 sequencing cycles. The number of sequencing cycles performed prior to cleavage may be selected based on the length of the barcode sequence or target sequence to be read, the desired sequencing depth, or the complexity of the multiplexed assay. In embodiments, performing a plurality of sequencing cycles prior to cleaving the first oligonucleotide enables the acquisition of sequence information sufficient to identify a target polynucleotide or to decode a barcode sequence associated with a biomolecule of interest. In embodiments, the sequence information obtained from the plurality of sequencing cycles is used to distinguish among a plurality of different target polynucleotides present in the cell or tissue. Following completion of the plurality of sequencing cycles, the first oligonucleotide is cleaved at the cleavable site to produce the first fragment and the second fragment, which are then removed as described herein, thereby preparing the sample for hybridization of the second oligonucleotide and subsequent detection of additional biomolecules.

In embodiments, the method includes sequencing the barcode (e.g., the barcode sequence or the barcode nucleotide). In embodiments, the method includes sequencing a plurality of barcodes in an optically resolved volume. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide barcode.

In embodiments, sequencing includes extending a first sequencing primer to generate a sequencing read comprising the first barcode sequence, or a portion thereof. In embodiments, sequencing includes extending a first sequencing primer to generate a sequencing read comprising the first barcode sequence, or a portion thereof, and extending a second sequencing primer to generate a sequencing read comprising the second barcode sequence. In embodiments, sequencing includes sequentially extending a plurality of sequencing primers (e.g., sequencing a first region of a target nucleic acid followed by sequencing a second region of a target nucleic acid, followed by sequencing N regions, where Nis the number of sequencing primers in the known sequencing primer set). In embodiments, sequencing includes generating a plurality of sequencing reads.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon TK. Error Correction Coding: Mathematical Methods and Algorithms. ed. 1st Wiley: 2005., each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule. In embodiments, sequencing generates a sequencing read.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) or acyclic nucleotide triphosphates to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) or acyclic nucleotide triphosphates to prevent further extension of the sequencing read product.

In embodiments, sequencing includes sequencing by synthesis, sequencing by binding, or sequencing by ligation. In embodiments, sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the amplification product.

In embodiments, prior to binding the polynucleotide probe, the method includes binding the specific binding agent to a target molecule in a cell or tissue. In embodiments, the targets are proteins or carbohydrates. In embodiments, the targets are proteins. In embodiments, the targets are carbohydrates. In embodiments, when the target are proteins and/or carbohydrates, the method includes contacting the proteins with a specific binding reagent, wherein the specific binding reagent comprises an oligonucleotide barcode. In embodiments, the specific binding reagent comprises an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 μm. A typical light sheet fluorescence microscope includes an sCMOS camera, a 2×/0.5 objective lens, and zoom microscope body (magnification range of ×0.63 to 6.3). For entire scanning of whole samples, the z-step interval is 5 or 10 μm, and for image acquisition in the regions of interest, an interval in the range of 2 to 5 μm may be used.

In embodiments, the method includes obtaining an image of the tissue sample. The imaging step may be performed with high-resolution techniques, such as fluorescence microscopy, which captures emitted signals from fluorescent particles introduced to specific surfaces of the tissue sample. In embodiments, and to ensure comprehensive visualization, images may be taken across multiple focal planes or cross-sections along the z-axis of the tissue, capturing features at various depths to enable identification of key structures and interfaces. Imaging parameters, including exposure time, gain settings, and focal depth, may be adjusted to optimize contrast and resolution, contributing to the clarity and precision of each acquired image. Obtaining detailed images allows for accurate localization of fluorescent particles relative to the tissue layers and supports subsequent computational analyses. These analyses may include edge detection via the Laplacian operator and variance calculations, instrumental in distinguishing boundaries and measuring distances within the tissue. The imaging process thereby provides a critical foundation for assessing the structural integrity, thickness, and spatial relationships among regions of interest within the tissue sample.

In embodiments, the imaging modality is capable of imaging an imaging area of about 1 cm² to about 10 cm², 1 cm² to about 5 cm², 5 cm² to about 10 cm², 10 cm² to about 30 cm², 30 cm² to about 60 cm², 60 cm² to about 90 cm², 90 cm² to about 120 cm², or more.

In embodiments, the collection of information (e.g., sequencing information and cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

In an aspect is provided a method for detecting plurality of proteins in a cell or tissue, the method including: (a) contacting the cell or tissue with a plurality of circularizable probes and forming a plurality of oligonucleotide complexes, wherein the cell or tissue includes the plurality of proteins and each protein includes an oligonucleotide, and forming an oligonucleotide complex includes binding a circularizable probe to the oligonucleotide; (b) circularizing each circularizable probe to form a plurality of circular polynucleotides, wherein each circular polynucleotide including a primer binding sequence and a barcode sequence; (c) amplifying the circular polynucleotides by rolling circle amplification to generate a plurality of amplification products; (d) hybridizing a first sequencing primer to a first subset of the amplification products, wherein the first sequencing primer includes a cleavable site, incorporating with a polymerase a first labeled nucleotide into the first sequencing primer, and detecting the first labeled nucleotide to identify a first subset of target proteins; (e) cleaving the first sequencing primer at the cleavable site to produce a first fragment and a second fragment, and removing the first fragment and the second fragment; and (f) hybridizing a second sequencing primer to a second subset of the amplification products, incorporating with a polymerase a second labeled nucleotide into the second sequencing primer, and detecting the second labeled nucleotide to identify a second subset of target proteins.

In embodiments, the method further includes contacting the cell or tissue with a stain. A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the first fluorescent dye. In embodiments, detecting includes directing an excitation light to the cell or tissue and detecting an emission light from the second fluorescent dye. In embodiments, detecting includes directing an excitation light to the cell or tissue and detecting an emission light from the stain. In embodiments, detecting includes directing an excitation light to the cell or tissue and detecting an emission light from a first fluorescent dye, a second fluorescent dye, and the stain.

### EXAMPLES

### Example 1. Transitioning Between Detection Rounds

Early disease diagnosis plays an important role in effective treatment. Clinical evidence based on a single analyte or single biomarker is typically not adequate for a confident diagnosis of a disease or monitoring treatment. While commercial single-plex techniques such as enzyme-linked immunosorbent assay (ELISA) (e.g., sandwich immunoassays) and biomarker kits can accurately detect a single analyte, the monitoring of more complex, multifactorial diseases such as cancer, autoimmune, and neurodegenerative diseases require the analysis of multiple biomarkers in order to confidently address the underlying disease (e.g., deciding best treatment options, tracking disease progression and response to therapy, and formulating prognoses). Multiplex detection methods, such as those described herein, confer several advantages over widely used single-plex assays including increased efficiency, greater output per sample volume, and higher throughput. For almost 50 years, immunoassays have allowed for sensitive and specific detection of analytes of interest in biological samples. Typical immunoassays include staining biomolecules, which include binding an antibody (Ab) to a target analyte and detecting the Ab directly (e.g., the Ab is labeled with a fluorophore, referred to as a primary antibody) or indirectly (i.e., a second labeled antibody binds to the first antibody and is detected). Staining is limited by spectral overlap.

Spectral overlap occurs when two or more fluorophores used to detect different targets emit similar wavelengths of light that overlap in the detection spectrum. The resulting light intensity signals for the different targets become muddled and make it difficult or impossible to distinguish between them. To minimize spectral overlap, different fluorophores with contrasting colors should be used for each target so that the individual spectral signals can be clearly distinguished. Generally, acceptable thresholds of spectral overlap for detection are set such that the emission peaks of the different fluorophores minimally overlap, e.g., less than 25% overlap. Because emission spectra have a variety of shapes, widths, and degrees of overlap, the degree of spectral overlap varies greatly. In order to maximize separation between fluorophores for multiplexing, it is important to choose dyes with distinct emission spectra and wavelengths that do not overlap with one another. Given the narrow range (e.g., typical emission wavelengths range from 400-700 nm) there are a finite number of fluorophores one can use. For example, detecting five targets requires five different fluorophores: (i) Alexa Fluor® 488 (ex/em wavelength of 495/520 nm); (ii) Cy®3 (ex/em wavelength of 559/573 nm), (iii) Cy®5 (ex/em wavelength of 649/670 nm); (iv) Cy®7 (ex/em wavelength of 759/780 nm), and (v) DyLight™ 649 (ex/em wavelength of 646/671 nm). Practically, this limits approaches to detect four to five biomolecules simultaneously. Strategies to overcome this limitation enabling multi-biomolecule detection include cyclical staining protocols, wherein after each round of staining the fluorophores are inactivated or the bound antibodies are removed. Various methods are used in biology and medicine to analyze different targets in biological samples. Proteins in histological sections and other cytological preparations may be examined using histochemistry, immunohistochemistry (IHC), or immunofluorescence (IF). Many of these techniques are limited in their ability to detect multiple targets simultaneously. For example, IHC and fluorescence-based western blotting typically detect only a few targets at a time due to constraints imposed by the fluorescence detection system.

Recent advances in antibody-based multiplexing have improved single-cell and subcellular spatial resolution, enabling the simultaneous imaging of multiple proteins within biological samples. While these methods can provide sensitive and specific detection, they are constrained by the limited spectral range available for fluorescence detection. Spectral overlap occurs when fluorophores emit light at similar wavelengths, making it difficult to distinguish between different targets. Spectral overlap necessitates the use of dyes with minimal emission overlap, often restricting the number of biomarkers that can be simultaneously detected to four or five, necessitating the development of alternative multiplexing strategies. Multiplexed fluorescence microscopy (MxIF) and Cyclic Immunofluorescence (CyCIF) were among the first approaches to utilize cyclic imaging for multiplexed protein detection, which involves cyclic imaging, where repeated rounds of staining, imaging, and bleaching of fluorophores increase multiplexing capacity, enabling the detection of dozens of analytes through iterative imaging cycles. However these methods can damage the sample and prolong the imaging process. As a result, detecting a large number of targets can become impractically lengthy and complex.

Singular Genomics has developed an antibody-oligonucleotide conjugate approach for multiplexed protein detection, integrating sequencing-based readout methods to overcome the limitations of fluorescence-based assays. In this method, antibodies conjugated to unique oligonucleotides bind to target proteins in biological samples. A circularizable probe hybridizes to the antibody-oligonucleotide conjugate, is ligated to form a closed circle, and undergoes rolling circle amplification (RCA) to generate a concatemer of repeated sequence motifs. These amplification products are subsequently detected through sequencing-by-synthesis (SBS), where fluorescently labeled nucleotides are incorporated and imaged in one or more cycles to decode the target identity (e.g., the oligonucleotide sequence conjugated to the antibody and/or an identifying barcode in the circularizable probe). To expand multiplexing capacity, different amplification products may be read out in batches by using multiple rounds of sequencing primers (e.g., 4-6 different sequencing primer rounds), allowing for the detection of a larger number of targets within a single sample.

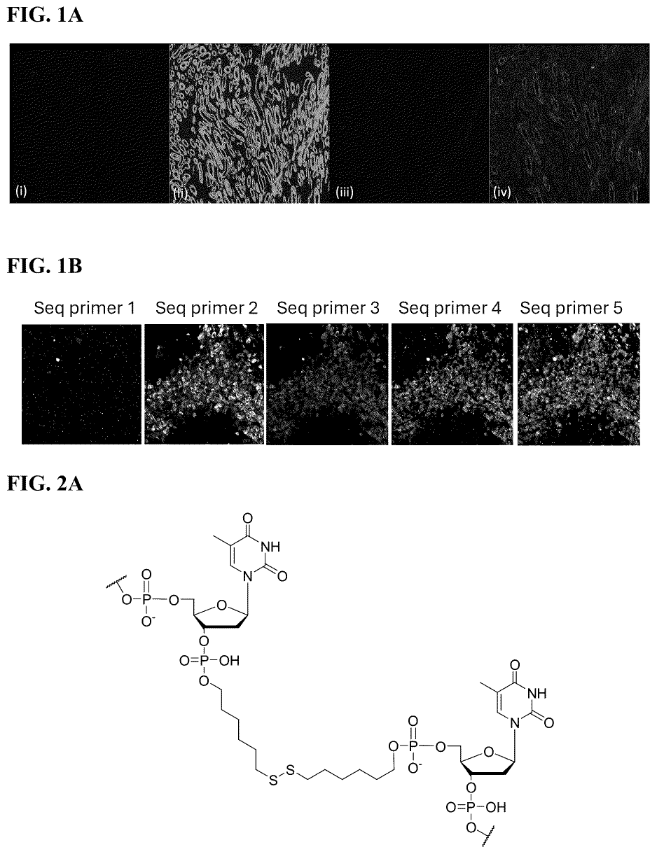

Despite its advantages in multiplexing, transitioning between sequencing primer rounds presents challenges, particularly in sequencing read termination and primer removal. A preferred method of termination includes incorporating a permanently terminated nucleotide, such as a dideoxynucleotide (ddNTP) or nucleotide analogue incapable of extension. However, inefficient termination can result in the incorporation of unintended nucleotides, leading to background signal and reduced sequencing accuracy. For example, a representative series of images is shown in FIG. 1A. Briefly, following sample preparation and amplification, a first image of the kidney is obtained (i), followed by incorporation of a labeled nucleotide (ii). The labeled nucleotide is incorporated into a sequencing primer bound to the amplification product. Following cleavage and incorporation of a terminated nucleotide (e.g., ddNTP), a third image is obtained (iii). One would expect that after incorporating an irreversibly terminated nucleotide incapable of extending, that upon exposure to additional labeled nucleotides nothing would be incorporated. However, as shown in FIG. 1A panel (iv), labeled nucleotides are detected, indicating the nucleotides are still binding to the polymerase complex during imaging. When sequencing over multiple rounds, for example 5 different rounds of sequencing, biomolecules are illuminated and detected in every round (FIG. 1). A method that enables efficient primer removal while maintaining sample integrity is critical for improving the accuracy, scalability, and reproducibility of this sequencing-based protein detection platform.

The present disclosure describes a method for improving multiplexed detection in biological samples by introducing a cleavable sequencing primer that facilitates efficient primer removal. For example, incorporating a cleavable site within the primer allows for controlled fragmentation, which reduces the melting temperature (Tm) of the resulting fragments upon cleavage. This decrease in thermal stability enables primer removal under standard wash conditions (e.g., 60° C.) without requiring significant temperature changes, additional enzymes, or harsh denaturing agents. By improving sequencing read termination, this approach enhances multiplexing capacity while maintaining compatibility with workflows for detecting both proteins and RNA in complex biological samples.

### Example 2. Evaluation of Cleavable Sequencing Primers in Tonsil and Kidney Tissues

To evaluate the performance of cleavable sequencing primers in a combined RNA and protein detection assay, experiments were conducted using tissue samples from tonsil and kidney. The study aimed to compare two primer designs, referred to as JAR1 and JAR2, to assess their efficiency in sequencing read termination and primer removal.

TABLE 1
Melting temperatures (Tm) of JAR1 cleavable primers. The Tm values in Celsius represent
the melting temperatures of the 5′ arm and the 3′ arm when hybridized to different nucleotides
(A, C, G, or T).

| | Tm | Tm | Tm | Tm | Tm |
|---|---|---|---|---|---|
| Primer | (5′ Arm) | (3′ Arm + A) | (3′ Arm + C) | (3′ Arm + G) | (3′ Arm + T) |
| S1c | 50.7 | 59.5 | 60.9 | 61.2 | 60.4 |
| S2c | 51.7 | 59.0 | 60.3 | 60.6 | 59.9 |
| S3c | 54.5 | 57.9 | 59.5 | 59.8 | 58.9 |
| S4c | 54.5 | 57.7 | 59.1 | 59.4 | 58.6 |
| S5c | 53.9 | 56.4 | 57.8 | 58.1 | 57.3 |

TABLE 2
Melting temperatures (Tm) of V2 cleavable primers. The Tm values in
Celsius represent the melting temperatures of the 5′ arm and the 3′ arm
when hybridized to different nucleotides (A, C, G, or T).

| | Tm | Tm | Tm | Tm | Tm |
|---|---|---|---|---|---|
| Primer | (5′ Arm) | (3′ Arm + A) | (3′ Arm + C) | (3′ Arm + G) | (3′ Arm + T) |
| S1cc | 57.4 | 55.0 | 56.7 | 57.0 | 56.1 |
| S2cc | 58.3 | 52.7 | 54.4 | 54.8 | 53.8 |
| S3cc | 57.9 | 54.4 | 56.1 | 56.5 | 55.4 |
| S4cc | 57.7 | 54.3 | 55.9 | 56.3 | 55.3 |
| S5cc | 57.1 | 53.4 | 54.9 | 55.2 | 54.3 |

Compared to the JAR1 primers in Table 1, the JAR2 primers exhibit lower Tm values for the 3′ arm, which may contribute to improved primer removal under standard wash conditions. For protein detection, antibody-oligonucleotide conjugates targeting CD8, CD4, and CD34 were applied to the healthy tonsil tissue sections at a concentration of 5 μg/mL and incubated for two hours at 37° C. to facilitate binding to their respective protein targets. Simultaneously, RNA detection was performed using a method described elsewhere (e.g., within U.S. Pat. No. 11,643,679, which is incorporated herein by reference in its entirety), in which a panel of padlock probes targeting 300 genes was hybridized to RNA molecules. In parallel, a set of probes complementary to the antibody-oligonucleotide conjugates was introduced to enable protein detection through the same amplification strategy. Hybridization was carried out overnight at 45° C. with probes at a concentration of 100 nM. Following hybridization, the probes were ligated, and rolling circle amplification (RCA) was performed to generate concatemeric amplicons for subsequent sequencing-based detection. RCA was carried out at 37° C. for four hours, enabling co-amplification of both RNA and protein targets within the same reaction. The amplification products were then subjected to sequencing to assess the efficiency of primer removal and read termination between the two primer designs. For this experiment, the sequencing primers are annealed at 60° C. for 10 minutes and a first image is obtained. Next, a single labeled nucleotide with a reversible terminator is incorporated at 60° C. and another image is obtained. The reversible terminator is removed. Next a modified nucleotide containing a non-extendable 3′ end is incorporated and another image is obtained. Finally, a solution of labeled nucleotides is incubated for 5 minutes at 60° C. and another image is obtained.

Following sequencing cycles, primer removal was assessed by quantifying residual signal from previously hybridized primers. Post-cleavage, the JAR2 primer fragments dissociated effectively at 61° C., reducing background fluorescence and minimizing interference in subsequent sequencing cycles. Following cleavage, the sequencing primer is fragmented into two smaller oligonucleotide segments, resulting in a lower melting temperature (Tm) compared to the intact primer. This reduction in Tm allows the cleaved fragments to dissociate under standard wash conditions at 61° C., facilitating efficient primer removal without requiring additional enzymatic or chemical treatments. The cleavable primer design is specifically implemented to enable primer dissociation within the standard sequencing workflow, ensuring that residual primers do not interfere with subsequent sequencing rounds.

Varying the cleavable site. To evaluate the impact of cleavage site positioning on primer removal efficiency and sequencing performance, a series of primer variants were designed with single or multiple cleavage sites at different locations within the sequence, reported in Table 3. The non-cleavable control (S1nc) was included to establish a baseline for primer retention under standard wash conditions. The S1dc primer, containing two cleavage sites, was designed to test whether multiple cleavage events further destabilize the primer, facilitating more efficient removal. The S1cc primer, with a cleavage site centrally positioned within the sequence, serves as the primary single-site cleavage design. Additional variants (S1c+15, S1c+11, S1c+7, and S1c+3) were generated with single cleavage sites progressively closer to the 3′ end, allowing assessment of how the cleavage position relative to the hybridized region influences primer dissociation.

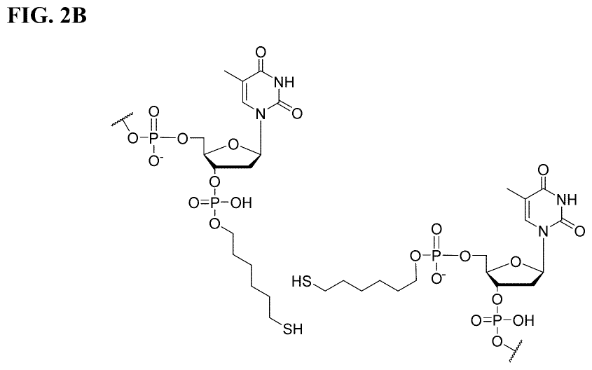

TABLE 3
Oligonucleotide sequence Table. The [SS-C6] denotes an
internucleosidic disulfide bond with a six-carbon spacer
within the primer sequence (see FIG. 2A) connecting the
5′ arm and the 3′ arm. As presented in the table the
sequences along the same row are connected to each other.

| | | | Cleavable | | |
|---|---|---|---|---|---|
| Ref No. | 5′ arm | | site | 3′ arm | |
| S1c | ATTTCATTCGAC | | [SS-C6] | GGTGCAGCCTCATTGT | |
| | ACCC | | | SEQ ID NO: 2 | |
| | SEQ ID NO: 1 | | | | |
| S2c | GTTTGAGTGAAG | | [SS-C6] | GTCCAACCCTTAGTGCGT | |
| | GCG | | | SEQ ID NO: 4 | |
| | SEQ ID NO: 3 | | | | |
| S3c | TTCGGCCAGAGT | | [SS-C6] | CGTGCAGCTTGCGT | |
| | TTAATG | | | SEQ ID NO: 6 | |
| | SEQ ID NO: 5 | | | | |
| S4c | GTACCCTGCGAT | | [SS-C6] | ACTCCAGCTGCGTAGT | |
| | CAG | | | SEQ ID NO: 8 | |
| | SEQ ID NO: 7 | | | | |
| S5c | CTGCTTGCATAG | | [SS-C6] | GGAATCTTGGTGCGACT | |
| | GGAG | | | SEQ ID NO: 10 | |
| | SEQ ID NO: 9 | | | | |
| S1cc | ATTTCATTCGAC | | [SS-C6] | TCGCAGCCTCATTGT | |
| | ACCCGG | | | SEQ ID NO: 12 | |
| | SEQ ID NO: 11 | | | | |
| S2cc | GTTTGAGTGAAG | | [SS-C6] | CAACCCTTAGTGCGT | |
| | GGTC | | | SEQ ID NO: 14 | |
| | SEQ ID NO: 13 | | | | |
| S3cc | TTCGGCCAGAGT | | [SS-C6] | GTTGCAGCTTGCGT | |
| | TTAATGC | | | SEQ ID NO: 16 | |
| | SEQ ID NO: 15 | | | | |
| S4cc | GTACCCTGCGAT | | [SS-C6] | TCCAGCTGCGTAGT | |
| | CAGAC | | | SEQ ID NO: 18 | |
| | SEQ ID NO: 17 | | | | |
| S5cc | CTGCTTGCATAG | | [SS-C6] | GAATCTTGGTGCGACT | |
| | GGAGG | | | SEQ ID NO: 20 | |
| | SEQ ID NO: 19 | | | | |
| S1nc | ATTTCATTCGACACCCGGGTCGAGCCTCATTGT | | | | |
| | SEQ ID NO: 21 | | | | |
| S1c + 15 | ATTTCATTCGAC | | [SS-C6] | TCGCAGCCTCATTGT | |
| | ACCCGG | | | SEQ ID NO: 23 | |
| | SEQ ID NO: 22 | | | | |
| S1c + 11 | ATTTCATTCGAC | | [SS-C6] | AGCCTCATTGT | |
| | ACCCGGC | | | SEQ ID NO: 25 | |
| | SEQ ID NO: 24 | | | | |
| S1c + 7 | ATTTCATTCGAC | | [SS-C6] | TCATTGT | |
| | ACCC | | | SEQ ID NO: 27 | |
| | SEQ ID NO: 26 | | | | |
| S1c + 3 | ATTTCATTCGAC | | [SS-C6] | TGT | |
| | ACCCGGCAT | | | SEQ ID NO: 29 | |
| | SEQ ID NO: 28 | | | | |
| RC1 | ATTTCATTCGAC | | [SS-C6] | TCGCAGCCTCATTGT | |
| | ACCCGG | | | SEQ ID NO: 31 | |
| | SEQ ID NO: 30 | | | | |
| RC2 | GTTTGAGTGAAG | | [SS-C6] | CAACCCTTAGTGCGT | |
| | GCGGTC | | | SEQ ID NO: 33 | |
| | SEQ ID NO: 32 | | | | |
| RC3 | TTCGGCCAGAGT | | [SS-C6] | GTTGCAGCTTGCGT | |
| | TTAATGC | | | SEQ ID NO: 35 | |
| | SEQ ID NO: 34 | | | | |
| RC4 | GTACCGTGCGAA | | [SS-C6] | TCCAGCTTGCGTAGT | |
| | TCAGAC | | | SEQ ID NO: 37 | |
| | SEQ ID NO: 36 | | | | |
| RC5 | CTGCTTGCATAG | | [SS-C6] | GAATCTTGGTGCGACT | |
| | GGAGG | | | SEQ ID NO: 39 | |
| | SEQ ID NO: 38 | | | | |
| RC6 | CGAGTTGGACA | | [SS-C6] | CCTCCCAAGGTTCCT | |
| | ATGATCG | | | SEQ ID NO: 41 | |
| | SEQ ID NO: 40 | | | | |
| S1dc | ATTTCATTCGA | [SS-C6] | CACCCGGTGC | [SS-C6] | AGCCTCATTGT |
| | SEQ ID NO: 42 | | SEQ ID NO: 43 | | SEQ ID NO: 44 |

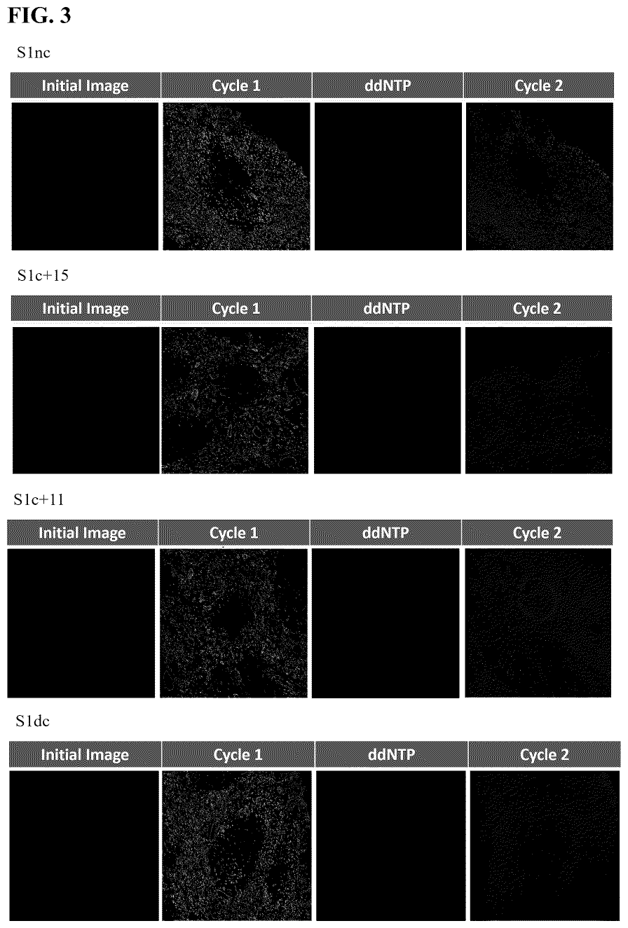

The performance of the cleavable primers was assessed by measuring residual signal in post-block extension to evaluate the efficiency of primer cleavage and removal. Primers S1c+15, S1C+11, as well as the double-cleavage primer (S1dc), exhibited little to no observable signal in post-block extension, indicating that cleavage and primer removal were highly efficient, relative to the control S1nc. See FIG. 3. Fluorescence intensity measurements from sequencing-by-synthesis and fluorescence in situ hybridization (FISH) revealed consistent signal levels among the S1c+15, S1c+11, S1cc, and the non-cleavable control (S1nc), suggesting that primer cleavage does not negatively impact overall signal intensity or sequencing efficiency.

The S1c+15 primer was further evaluated in a multiplex protein assay to assess its performance in a complex sequencing workflow. Antibody-oligonucleotide (Ab-O) conjugates targeting CD3, CD20, ATPase, HLADR, CD8, and CD45RA were incubated according to the previously described protocol. Simultaneously, a transcript panel targeting 300 genes and circularizable probes for the respective Ab-O targets were introduced, hybridized, circularized, and amplified.

Sequencing was performed in two rounds, each driven by a distinct sequencing primer. In the first round, the S1c+15 primer was used to detect CD3 (incorporated labeled A), CD20 (incorporated labeled T), ATPase (incorporated labeled G), and HLADR (incorporated labeled C). Following this, the S1c primer was used in the second round to detect CD8 (incorporated labeled A) and CD45RA (incorporated labeled G). Between sequencing rounds, the S1c+15 primer was cleaved and removed before the incorporation of labeled nucleotides in the second round, ensuring that residual primers from the first round did not interfere with subsequent sequencing. Each round consisted of multiple sequencing cycles, with each cycle corresponding to an individual nucleotide incorporation event. The number of incorporation events per cycle typically ranges from 1 to 150, depending on the sequencing conditions and desired sequence read length.

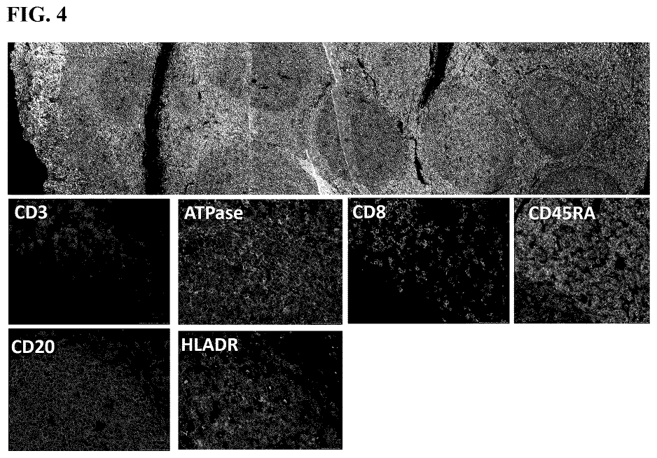

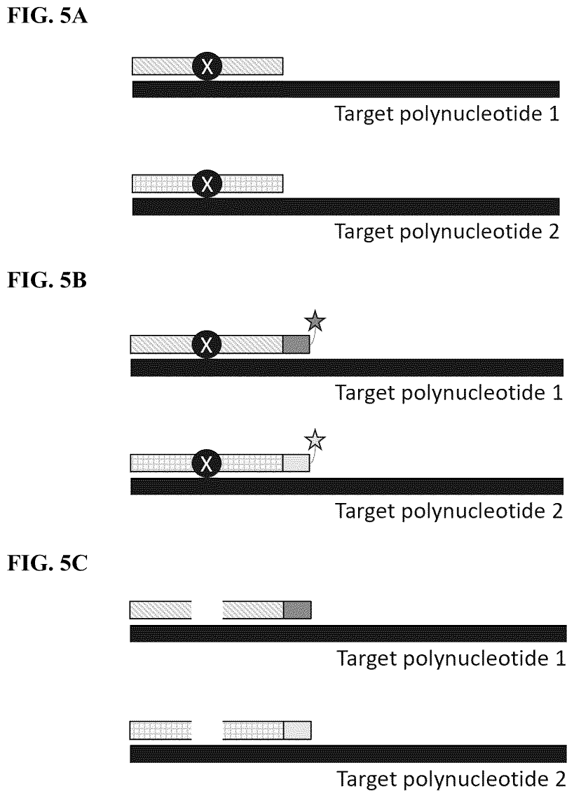

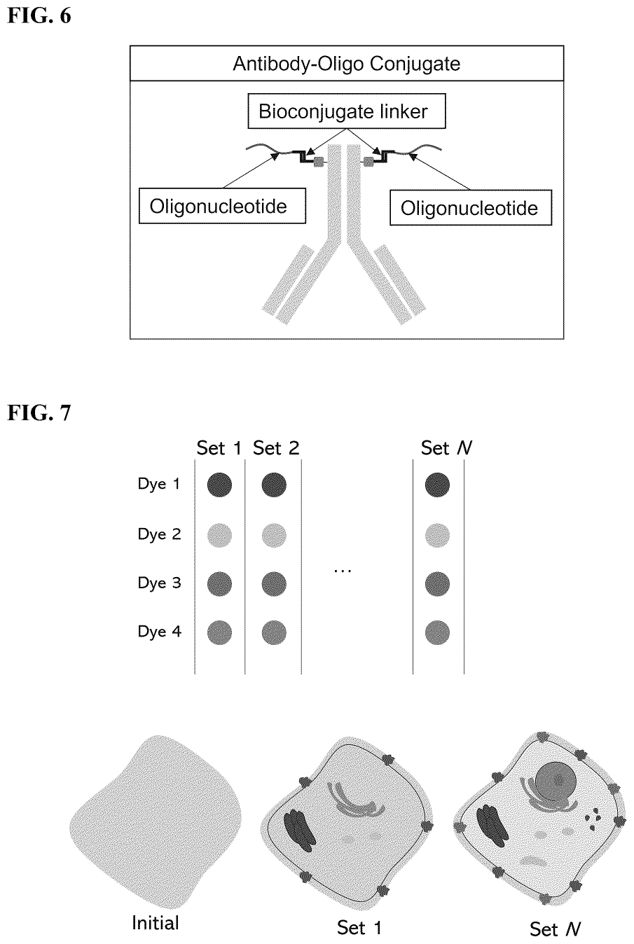

Several targets exhibited well-preserved morphology in tonsil tissue, as reported in FIG. 4, suggesting that the assay conditions effectively maintained tissue integrity. For these targets, the observed morphology represents the highest quality achieved to date using a 4-hour co-amplification protocol for simultaneous RNA and protein detection.

### Example 3. Detection Strategy for Multiplexed Protein Sequencing

In this multiplexed protein sequencing approach, each column in the detection scheme represents a distinct sequencing primer round (Seq Primer 1, Seq Primer 2, Seq Primer 3, Seq Primer 4, and Seq Primer 5). The sequencing primers used in each round may contain a cleavable site as described herein, allowing for controlled cleavage and removal before the next round of sequencing begins. This step ensures that residual primers from previous rounds do not interfere with subsequent sequencing cycles.

TABLE 4
Example Detection Scheme for Multiplexed Protein
Sequencing. Five rounds of detection are used, wherein
each round is initiated by a unique sequencing primer.

| | Seq | Seq | Seq | Seq | Seq |
|---|---|---|---|---|---|
| | Primer 1 | Primer 2 | Primer 3 | Primer 4 | Primer 5 |
| C (Cytosine) | CD4 | CD56 | CD3e | CD68 | MsIgG2a |
| T (Thymine) | CD31 | FoxP3 | CD45RA | HLA-DR | ATPase |
| G (Guanine) | PD-L1 | CD11c | PD1 | PanCK | aSMA |
| A (Adenine) | CTLA-4 | Ki67 | CD20 | CD8 | MsIgG1 |

During each sequencing round, proteins are identified based on the incorporation of a specific labeled nucleotide (cytosine (C), thymine (T), guanine (G), or adenine (A)) which corresponds to a particular protein target. For example, in the first sequencing round using primer Seq Primer 1, proteins CD4, CD31, PD-L1, and CTLA-4 are detected based on the incorporation of C, T, G, and A, respectively. Following incorporating of a labeled nucleotide and imaging, the Seq Primer 1 primer is cleaved and removed, allowing for the introduction of primer Seq Primer 2 for the second sequencing round. These detection cycles are repeated across additional rounds, with each primer enabling the detection of a different subset of proteins.

By structuring the sequencing rounds in this manner, the method enables efficient multiplexed protein detection within a single sample. The cleavable primer design facilitates seamless transitions between sequencing rounds, minimizing background signal and ensuring accurate protein identification. The sequential primer use, combined with barcode-based target identification, supports robust multiplexing in spatial biology applications.

What is claimed is:

1. A method of detecting biomolecules in a cell or tissue, said method comprising:
(a) hybridizing a first oligonucleotide to a first target polynucleotide in the cell or tissue, wherein the first oligonucleotide comprises a cleavable site, and detecting a first detectable label associated with the first oligonucleotide;
(b) cleaving the first oligonucleotide at the cleavable site to produce a first fragment and a second fragment, wherein each of the first fragment and the second fragment has a melting temperature lower than a melting temperature of the first oligonucleotide, and removing the first fragment and the second fragment; and
(c) hybridizing a second oligonucleotide to a second target polynucleotide in the cell or tissue, and detecting a second detectable label associated with the second oligonucleotide.

2. The method of claim 1, wherein (a), (b), and (c) occur at substantially the same temperature.

3. The method of claim 2, wherein (a), (b), and (c) occur at about 55° C. to about 65° C.

4. The method of claim 2, wherein (a), (b), and (c) occur at about 60° C.

5. The method of claim 1, wherein the first detectable label is a fluorescent label covalently attached to the first oligonucleotide.

6. The method of claim 1, wherein the first oligonucleotide is a sequencing primer and detecting the first detectable label comprises binding a labeled nucleotide to the first oligonucleotide with a polymerase and detecting the labeled nucleotide.

7. The method of claim 1, wherein the first oligonucleotide is a sequencing primer and detecting the first detectable label comprises incorporating a labeled nucleotide into the first oligonucleotide with a polymerase and detecting the labeled nucleotide.

8. The method of claim 7, wherein the labeled nucleotide comprises a reversible terminator moiety and a fluorescent label covalently linked to the nucleotide via a cleavable linker.

9. The method of claim 7, further comprising incorporating a plurality of labeled nucleotides into the first oligonucleotide in a plurality of sequencing cycles prior to (b).

10. The method of claim 1, wherein the cleavable site comprises a disulfide scissile internucleosidic linkage.

11. The method of claim 1, wherein the cleavable site comprises a deoxyuracil triphosphate (dUTP), a deoxy-8-oxo-guanine triphosphate (d-8-oxoG), a methylated nucleotide, or a ribonucleotide.

12. The method of claim 1, wherein the first oligonucleotide comprises two or more cleavable sites.

13. The method of claim 1, wherein cleaving the first oligonucleotide comprises contacting the first oligonucleotide with a reducing agent.

14. The method of claim 1, further comprising repeating (a), (b), and (c) in series to detect additional biomolecules.

15. The method of claim 1, wherein the first target polynucleotide and the second target polynucleotide are each formed via rolling circle amplification of a circularized probe.

16. The method of claim 15, wherein the circularized probe is hybridized to an oligonucleotide conjugated to a specific binding agent, wherein the specific binding agent is an antibody, single-chain Fv fragment (scFv), antibody fragment-antigen binding (Fab), affimer, or an aptamer.

17. The method of claim 16, wherein the circularized probe comprises a barcode sequence.

18. A system for detecting biomolecules in a cell or tissue, the system comprising:
a substrate comprising a channel, wherein the cell or tissue is immobilized within the channel;
a first oligonucleotide comprising a cleavable site, wherein cleaving the first oligonucleotide at the cleavable site produces a first fragment and a second fragment, each having a melting temperature lower than a melting temperature of the first oligonucleotide;
a second oligonucleotide;
a cleaving agent configured to cleave the cleavable site of the first oligonucleotide;
a polymerase; and
a plurality of labeled nucleotides,
wherein the system is configured to sequentially: (i) hybridize the first oligonucleotide to a first target polynucleotide in the cell or tissue and detect a signal associated with the first oligonucleotide, (ii) cleave the first oligonucleotide at the cleavable site and remove the first fragment and the second fragment, and (iii) hybridize the second oligonucleotide to a second target polynucleotide in the cell or tissue and detect a signal associated with the second oligonucleotide.

19. The system of claim 18, further comprising a fluidic system in fluid communication with the channel, wherein the fluidic system is configured to deliver the first oligonucleotide, the second oligonucleotide, the cleaving agent, the polymerase, and the plurality of labeled nucleotides to the channel.

20. The system of claim 18, further comprising an imaging system configured to detect the signal associated with the first oligonucleotide and the signal associated with the second oligonucleotide.

21. The system of claim 20, wherein the imaging system comprises a fluorescence excitation source.

22. The system of claim 18, wherein the cleaving agent comprises a reducing agent.

23. The system of claim 22, wherein the reducing agent comprises tris(2-carboxyethyl)phosphine (TCEP) or tris(3-hydroxypropyl)phosphine (THPP).

24. The system of claim 18, wherein the substrate comprises a glass slide.

25. The system of claim 18, wherein the substrate comprises a flow cell.

26. The system of claim 18, wherein the system is configured to maintain a substantially constant temperature during (i), (ii), and (iii).

27. A method of detecting a plurality of proteins in a cell or tissue, the method comprising:
(a) contacting the cell or tissue with a plurality of circularizable probes and forming a plurality of oligonucleotide complexes, wherein the cell or tissue comprises the plurality of proteins and each protein comprises an oligonucleotide, and forming an oligonucleotide complex comprises binding a circularizable probe to the oligonucleotide;
(b) circularizing each circularizable probe to form a plurality of circular polynucleotides, wherein each circular polynucleotide comprising a primer binding sequence and a barcode sequence;
(c) amplifying the circular polynucleotides by rolling circle amplification to generate a plurality of amplification products;
(d) hybridizing a first sequencing primer to a first subset of the amplification products, wherein the first sequencing primer comprises a cleavable site, incorporating with a polymerase a first labeled nucleotide into the first sequencing primer, and detecting the first labeled nucleotide to identify a first subset of target proteins;
(e) cleaving the first sequencing primer at the cleavable site to produce a first fragment and a second fragment, and removing the first fragment and the second fragment; and
(f) hybridizing a second sequencing primer to a second subset of the amplification products, incorporating with a polymerase a second labeled nucleotide into the second sequencing primer, and detecting the second labeled nucleotide to identify a second subset of target proteins.
